SEQUENCE LISTING (43 sequences)

SEQ ID NO: 1 - protein, 9 aa, Artificial Sequence
  Artificial sequence derived from human and animal adenovirus peptide sequences for binding to endothelial cell surface molecules
CPDLHHHMC

SEQ ID NO: 2 - protein, 9 aa, Artificial Sequence
  Artificial sequence derived from human and animal adenovirus peptide sequences for binding to endothelial cell surface molecules
CLGQHAFTC

SEQ ID NO: 3 - protein, 9 aa, Artificial Sequence
  Artificial sequence derived from human and animal adenovirus peptide sequences for binding to endothelial cell surface molecules
CSSNTAPHC

SEQ ID NO: 4 - protein, 9 aa, Artificial Sequence
  Artificial sequence derived from human and animal adenovirus peptide sequences for binding to endothelial cell surface molecules
CHVLPNGNC

SEQ ID NO: 5 - protein, 10 aa, Artificial Sequence
  Artificial sequence derived from human and animal adenovirus peptide sequences for binding to endothelial cell surface molecules
CKPGIYPSLC

SEQ ID NO: 6 - protein, 9 aa, Artificial Sequence
  Artificial sequence derived from human and animal adenovirus peptide sequences for binding to endothelial cell surface molecules
CQTARTPAC

SEQ ID NO: 7 - protein, 9 aa, Artificial Sequence
  Artificial sequence derived from human and animal adenovirus peptide sequences for binding to endothelial cell surface molecules
CNQSQPKHC

SEQ ID NO: 8 - protein, 9 aa, Artificial Sequence
  Artificial sequence derived from human and animal adenovirus peptide sequences for binding to endothelial cell surface molecules
CTPSKISVC

SEQ ID NO: 9 - protein, 9 aa, Artificial Sequence
  Artificial sequence derived from human and animal adenovirus peptide sequences for binding to endothelial cell surface molecules
CVSPGPRLC

SEQ ID NO: 10 - protein, 9 aa, Artificial Sequence
  Artificial sequence derived from human and animal adenovirus peptide sequences for binding to endothelial cell surface molecules
CYALSGVPC

SEQ ID NO: 11 - protein, 9 aa, Artificial Sequence
  Artificial sequence derived from human and animal adenovirus peptide sequences for binding to endothelial cell surface molecules
CKHPPQPFC

SEQ ID NO: 12 - protein, 9 aa, Artificial Sequence
  Artificial sequence derived from human and animal adenovirus peptide sequences for binding to endothelial cell surface molecules
CHQSKPLLC

SEQ ID NO: 13 - protein, 9 aa, Artificial Sequence
  Artificial sequence derived from human and animal adenovirus peptide sequences for binding to endothelial cell surface molecules
CPGPFSNWC

SEQ ID NO: 14 - protein, 9 aa, Artificial Sequence
  Artificial sequence derived from human and animal adenovirus peptide sequences for binding to endothelial cell surface molecules
CPHKTHLPC

SEQ ID NO: 15 - protein, 9 aa, Artificial Sequence
  Artificial sequence derived from human and animal adenovirus peptide sequences for binding to endothelial cell surface molecules
CVFPLSHYC

SEQ ID NO: 16 - protein, 9 aa, Artificial Sequence
  Artificial sequence derived from human and animal adenovirus peptide sequences for binding to endothelial cell surface molecules
CNMIAPSSC

SEQ ID NO: 17 - protein, 9 aa, Artificial Sequence
  Artificial sequence derived from human and animal adenovirus peptide sequences for binding to endothelial cell surface molecules
CTLGMQFQC

SEQ ID NO: 18 - protein, 9 aa, Artificial Sequence
  Artificial sequence derived from human and animal adenovirus peptide sequences for binding to endothelial cell surface molecules
CTNPTGMLC

SEQ ID NO: 19 - protein, 9 aa, Artificial Sequence
  Artificial sequence derived from human and animal adenovirus peptide sequences for binding to endothelial cell surface molecules
CSNMAPRSC

SEQ ID NO: 20 - protein, 9 aa, Artificial Sequence
  Artificial sequence derived from human and animal adenovirus peptide sequences for binding to endothelial cell surface molecules
CSMAPNMSC

SEQ ID NO: 21 - protein, 9 aa, Artificial Sequence
  Artificial sequence derived from human and animal adenovirus peptide sequences for binding to endothelial cell surface molecules
CSDLTMEAC

SEQ ID NO: 22 - protein, 9 aa, Artificial Sequence
  Artificial sequence derived from human and animal adenovirus peptide sequences for binding to endothelial cell surface molecules
CPWPYKYSC

SEQ ID NO: 23 - protein, 9 aa, Artificial Sequence
  Artificial sequence derived from human and animal adenovirus peptide sequences for binding to endothelial cell surface molecules
CFGGNFHRC

SEQ ID NO: 24 - protein, 9 aa, Artificial Sequence
  Artificial sequence derived from human and animal adenovirus peptide sequences for binding to endothelial cell surface molecules
CLTTSQQTC

SEQ ID NO: 25 - protein, 9 aa, Artificial Sequence
  Artificial sequence derived from human and animal adenovirus peptide sequences for binding to endothelial cell surface molecules
CTANSGSFC

SEQ ID NO: 26 - protein, 9 aa, Artificial Sequence
  Artificial sequence derived from human and animal adenovirus peptide sequences for binding to endothelial cell surface molecules
CQEPLDESC

SEQ ID NO: 27 - protein, 9 aa, Artificial Sequence
  Artificial sequence derived from human and animal adenovirus peptide sequences for binding to endothelial cell surface molecules
CQMSMFARC

SEQ ID NO: 28 - protein, 9 aa, Artificial Sequence
  Artificial sequence derived from human and animal adenovirus peptide sequences for binding to endothelial cell surface molecules
CPLTPKAYC

SEQ ID NO: 29 - protein, 9 aa, Artificial Sequence
  Artificial sequence derived from human and animal adenovirus peptide sequences for binding to endothelial cell surface molecules
CNNSHTALC

SEQ ID NO: 30 - protein, 9 aa, Artificial Sequence
  Artificial sequence derived from human and animal adenovirus peptide sequences for binding to endothelial cell surface molecules
CLSSDITLC

SEQ ID NO: 31 - protein, 9 aa, Artificial Sequence
  Artificial sequence derived from human and animal adenovirus peptide sequences for binding to endothelial cell surface molecules
CLTHGPKYC

SEQ ID NO: 32 - protein, 9 aa, Artificial Sequence
  Artificial sequence derived from human and animal adenovirus peptide sequences for binding to endothelial cell surface molecules
CLGKDLRTC

SEQ ID NO: 33 - protein, 9 aa, Artificial Sequence
  Artificial sequence derived from human and animal adenovirus peptide sequences for binding to endothelial cell surface molecules
CAPKTHPLC

SEQ ID NO: 34 - protein, 9 aa, Artificial Sequence
  Artificial sequence derived from human and animal adenovirus peptide sequences for binding to endothelial cell surface molecules
CPTGLMKYC

SEQ ID NO: 35 - protein, 9 aa, Artificial Sequence
  Artificial sequence derived from human and animal adenovirus peptide sequences for binding to endothelial cell surface molecules
CTWKAPLQC

SEQ ID NO: 36 - protein, 9 aa, Artificial Sequence
  Artificial sequence derived from human and animal adenovirus peptide sequences for binding to endothelial cell surface molecules
CSHILGPSC

SEQ ID NO: 37 - protein, 9 aa, Artificial Sequence
  Artificial sequence derived from human and animal adenovirus peptide sequences for binding to endothelial cell surface molecules
CLSTSQYSC

SEQ ID NO: 38 - DNA, 34 bp, Artificial Sequence
  Artificial oligo sequence derived from human and animal adenovirus peptide sequences for binding to endothelial cell surface molecules
gatcaatgtcctgacctacaccaccacatgtgtt

SEQ ID NO: 39 - DNA, 34 bp, Artificial Sequence
  Artificial oligo sequence derived from human and animal adenovirus peptide sequences for binding to endothelial cell surface molecules
gtacaacacatgtggtggtgtaggtcaggacatt

SEQ ID NO: 40 - DNA, 33 bp, Artificial Sequence
  Artificial oligo sequence derived from human and animal adenovirus peptide sequences for binding to endothelial cell surface molecules
ggcctgtcctgatcttcatcatcatatgtgtgc

SEQ ID NO: 41 - DNA, 33 bp, Artificial Sequence
  Artificial oligo sequence derived from human and animal adenovirus peptide sequences for binding to endothelial cell surface molecules
ggccgcacacatatgatgatgaagatcaggaca

SEQ ID NO: 42 - protein, 14 aa, Artificial Sequence
  Artificial peptide sequence derived from human and animal adenovirus peptide sequences for binding to endothelial cell surface molecules
KLAKLAKKLAKLAK

SEQ ID NO: 43 - protein, 9 aa, Artificial Sequence
  Consensus sequence for SEQ ID NOs 1-37, derived from human and animal adenovirus peptide sequences for binding to endothelial cell surface molecules
CXXPTPPXC

(19) United States
(12) Patent Application Publication
(10) Pub. No.: US 2006/0223756 A1
(43) Pub. Date: Oct. 5, 2006

(54) Endothelial cell specifically binding peptides

(76) Inventors: Gene Liau, Wayland, MA (US); Steingrimur Stefansson, Gaithersburg, MD (US); Joseph Su, Germantown, MD (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/537,847

(22) PCT Filed: Dec. 17, 2003
(86) PCT No.: PCT/EP03/14407

§ 371 Date: Nov. 14, 2005

Related U.S. Application Data
(60) Provisional application No. 60/434,258, filed on Dec. 18, 2002.

(51) Int. Cl. A61K 38/10; A61K 38/08; C07K 7/08; C07H 21/04; C12P 21/04

(52) U.S. Cl. 514/16; 435/69.7; 435/456; 435/325; 514/17; 530/328; 530/329; 530/330; 536/23.5

(57) ABSTRACT

The present invention relates to peptides that specifically bind to endothelial cells. The peptides can be incorporated into gene delivery vector particles and can also direct therapeutic agents, including proteins such as growth factors and cytokines as well as small molecules. The vector particles, peptides, or small molecules can be used for the treatment of cancer and cardiovascular diseases such as ischemic heart disease, peripheral limb disease, vein graft stenosis and restenosis.

### BACKGROUND OF THE INVENTION

The present invention relates generally to the targeting of therapeutic substances to specific cells. The invention is more particularly related to targeting molecules, e.g., peptides, for use in delivering substances to endothelial cells. Such targeting molecules may be used in a variety of therapeutic procedures. More specifically, the present invention is directed to peptides which specifically bind to endothelial cells. The peptides can be incorporated into gene delivery vehicles and can also direct therapeutic agents, including proteins (such as growth factors and cytokines) as well as small molecules (such as drugs and other therapeutic agents). The targeting vectors, peptides, or small molecules can be used for the treatment of various disorders, including cancer, diabetic retinopathy, macular degeneration, rheumatoid arthritis, psoriasis, plaque rupture, restenosis, ischemic vascular diseases, wound healing, congestive heart failure, myocardial ischemia, reperfusion injury, peripheral arterial diseases, obesity and cardiovascular diseases such as ischemic heart disease, peripheral limb disease, vein graft stenosis and restenosis.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

Although the effect of a particular pathology often is manifest throughout the body of the afflicted person, generally, the underlying pathology may affect only a single organ, tissue or cell type. In many cases, drugs are the treatment of choice for a patient suffering a particular disease. Gene therapy is a second option for treating a patient suffering a particular disease. Improving the delivery of drugs and other agents to target tissues has been the focus of considerable research for many years. Most agents currently administered to a patient parenterally are not targeted, resulting in systemic delivery of the agent to cells and tissues of the body where it is unnecessary, and often undesirable. This may result in adverse drug side effects, and often limits the dose of a drug (e.g., cytotoxic agents and other anti-cancer or anti-viral drugs) that can be administered. By comparison, although oral administration of drugs is generally recognized as a convenient and economical method of administration, oral administration can result in either (a) uptake of the drug through the epithelial barrier, resulting in undesirable systemic distribution, or (b) temporary residence of the drug within the gastrointestinal tract. Accordingly, a major goal has been to develop methods for specifically targeting agents to cells and tissues that may benefit from the treatment, and to avoid the general physiological effects of inappropriate delivery of such agents to other cells and tissues.

Efforts have been made to increase the target specificity of various drugs and gene delivery vehicles. In some cases, a particular cell type present in a diseased tissue or organ may express a unique cell surface marker. In such a case, an antibody can be raised against the unique cell surface marker and a drug can be linked to antibody (see, e.g., Ferkol et al., 2000). Upon administration of the drug/antibody complex to the patient, the binding of the antibody to the cell surface marker results in the delivery of a relatively high concentration of the drug to the diseased tissue or organ. Similar methods can be used where a particular cell type in the diseased organ expresses a unique cell surface receptor or a ligand for a particular receptor. In these cases, the drug can be linked to the specific ligand, such as a peptide, or to the receptor, respectively, thus providing a means to deliver a relatively high concentration of the drug to the diseased organ (see, e.g., Ruoslahti and Rajotte, 2000; WO 98/44938; WO 00/06195).

While linking a drug to a molecule that homes to a particular cell type present in a diseased organ or tissue provides significant advantages for treatment, there is a need to identify specific target cell markers that are expressed in only one or a few tissues or organs and to identify molecules that specifically interact with such markers. Various cell types can express unique markers and, therefore, provide potential targets for organ homing molecules. Endothelial cells, for example, which line the internal surfaces of blood vessels, can have distinct morphologies and biochemical markers in different tissues. The blood vessels of the lymphatic system, for example, express various adhesion proteins that serve to guide lymphocyte homing. For example, endothelial cells present in lymph nodes express a cell surface marker that is a ligand for L-selectin and endothelial cells in Peyer's patch venules express a ligand for the α₄β₇ integrin.

The capabilities to introduce a particular foreign or native gene sequence into a mammal and to control the expression of that gene are of substantial value in the fields of medical and biological research. Such capabilities provide a means for studying gene regulation and for designing a therapeutic basis for the treatment of disease. In addition to introducing the gene into mammals, providing expression of the gene specifically at the site of interest can be a challenge. Methods have been developed to deliver DNA to target cells by capitalizing on indigenous cellular pathways of macromolecular transport. In this regard, gene transfer has been accomplished via the receptor-mediated endocytosis pathway employing molecular conjugate vectors.

Most adenoviral serotypes utilize the coxsackie:adenovirus receptor (CAR), which is an integral membrane protein of unknown function other than binding adenovirus and group B coxsackie viruses (Bergelson et al., 1997). Adenovirus binding to CAR occurs via the fiber knob (Stevenson et al., 1995; Henry et al., 1994). Following fiber-mediated cell attachment, the penton base can bind to αᵥβ₃ and αᵥβ₅ integrin co-receptors via a RGD motif and potentiate internalization (Bai et al., 1993; Nemerow and Stewart, 1999). Molecular retargeting of adenovirus particles is hypothesized to increase the number of viral ligand-receptor interactions on the target cell membrane as well as the number of viral particles translocated to the cytoplasm of the targeted cells. The adenovirus fiber carboxy-terminus and the HI loop present in the fiber knob are examples of sites for the incorporation of peptide motifs specifically recognized by cell surface receptors expressed by the target cells. An adenovirus having an HI loop modified to contain a cyclic RGD motif was found to have enhanced gene delivery to veins (Hay et al., 2001).

Retroviral vectors are also used in gene therapy. The tropism of retroviral vector particles are also being modified by the insertion of short peptide ligands at multiple locations in the envelope. For example, Moloney murine leukemia virus envelope derivatives bearing short peptide ligands for gastrin-releasing protein and human epidermal growth factor receptors have been prepared (Gollan and Green, 2002). Pseudotyped viruses containing these chimeric envelope derivatives selectively transducer human cancer cell lines that overexpress the cognate receptor. A retrovirus targeting the gastrin-releasing protein receptor can deliver the thymidine kinase gene to human melanoma and breast cancer cells, which are killed by the subsequent addition of ganciclovir.

Vascular graft stenosis is a major complication after coronary artery bypass grafting. Surgical therapeutic approaches can utilize autologous saphenous veins or internal mammary arteries. Arterial grafts have a higher patency rate than venous grafts (Loop et al., 1986; Cameron et al., 1996). Thrombotic mechanisms are involved in the early occlusions (Yang et al., 1991) whereas late occlusions are the result of neointima formation and progression of the atherosclerotic plaque in the grafted vessels (Angelini and Newby, 1989; Kalan and Roberts, 1990). Gene therapy, specifically adenoviral-mediated delivery of transgenes, is a strategy currently being pursued to prevent bypass graft neointimal hyperplasia (Cable et al., 1999). Various therapeutic transgenes including nitric oxide synthase and matrix metalloproteinases have been evaluated in preclinical interpositional grafting models and have demonstrated efficacy in the reduction of neointima formation (Newby and Baker, 1999).

Thus, a need exists to develop peptides which specifically bind to endothelial cells. The present invention satisfies this need and provides related advantages as well.

### SUMMARY OF THE INVENTION

The present invention relates generally to the targeting of therapeutic substances to specific cells. The invention is more particularly related to targeting molecules, e.g., peptides, for use in delivering substances to endothelial cells. Such targeting molecules may be used in a variety of therapeutic procedures. More specifically, the present invention is directed to peptides which specifically bind to endothelial cells. The peptides can be incorporated into gene delivery vehicles and can also direct therapeutic agents, including proteins (such as growth factors and cytokines) as well as small molecules (such as drugs, radionuclides and other therapeutic agents). The targeting vectors, peptides, or small molecules can be used for the treatment of cancer, diabetic retinopathy, macular degeneration, rheumatoid arthritis, psoriasis, plaque rupture, restenosis, ischemic vascular diseases, wound healing, congestive heart failure, myocardial ischemia, reperfusion injury, peripheral arterial diseases, obesity and cardiovascular diseases such as ischemic heart disease, peripheral limb disease, vein graft stenosis and restenosis. Thus in one embodiment, the present invention provides a peptide which specifically binds to endothelial cells.

In a second embodiment, the present invention provides a targeting molecule linked to at least one biological agent, wherein the targeting molecule comprises a peptide which is specific for endothelial cells, including those peptides described herein. The biological agent includes, but is not limited to, radionuclides, drugs, peptides, proteins, nucleic acids, gene delivery vectors, liposomes and the like. In a third embodiment, the present invention provides a pharmaceutical composition comprising a targeting molecule linked to at least one biological agent, as described above, in combination with a pharmaceutically acceptable carrier.

In a third embodiment, the present invention provides a pharmaceutical composition comprising a targeting molecule linked to at least one biological agent, as described above, in combination with a pharmaceutically acceptable carrier.

In a fourth embodiment, the present invention provides methods for treating a patient afflicted with a disease, disorder or condition associated with endothelial cells, comprising administering to a patient a pharmaceutical composition as described above. Such diseases, disorders or conditions include, but are not limited to, cancer, diabetic retinopathy, macular degeneration, rheumatoid arthritis, psoriasis, plaque rupture, restenosis, ischemic vascular diseases, wound healing, congestive heart failure, myocardial ischemia, reperfusion injury, peripheral arterial diseases, obesity and cardiovascular diseases such as ischemic heart disease, peripheral limb disease, vein graft stenosis and restenosis. In a fifth embodiment, the present invention provides methods for inhibiting the development in a patient of a disease, disorder or condition associated with endothelial cells, such as those described above, comprising administering to a patient a pharmaceutical composition as described above.

### BRIEF DESCRIPTION OF THE FIGURES

FIG. 1 shows the plasmids used to generate Av3nBgPD1. FIG. 1A. p5FloxHRFPD1 contains the coding sequence of the modified fiber containing the PD1 peptide in the fiber HI loop. The 6 KB SpeI/PacI fragment is isolated and cloned into pNDSQ3.1 to generate pNDSQ3.1PD1. FIG. 1B. pNDSQ3.1PD1 contains the right hand portion of the adenovirus serotype 5 genome. The encoded fiber is modified to contain the PD1 peptide in the HI loop of the knob.

### DETAILED DESCRIPTION OF THE INVENTION

The present invention relates generally to the targeting of therapeutic substances to specific cells. The invention is more particularly related to targeting molecules, e.g., peptides, for use in delivering substances to endothelial cells. Such targeting molecules may be used in a variety of therapeutic procedures. More specifically, the present invention is directed to peptides which specifically bind to endothelial cells. The peptides can be incorporated into gene delivery vehicles and can also direct agents, including proteins (such as growth factors and cytokines) as well as small molecules (such as drugs, radionuclides and other therapeutic agents). The targeting vectors, peptides, or small molecules can be used to target cells in vivo or in vitro. Targeting of endothelial cells can be used to deliver genes, peptides, and small molecules for the many purposes including studying cellular processes, marking cells or for therapeutic purposes. The targeting vectors, peptides, or small molecules can be used for the treatment of cancer, diabetic retinopathy, macular degeneration, rheumatoid arthritis, psoriasis, plaque rupture, restenosis, ischemic vascular diseases, wound healing, congestive heart failure, myocardial ischemia, reperfusion injury, peripheral arterial diseases, obesity and cardiovascular diseases such as ischemic heart disease, peripheral limb disease, vein graft stenosis and restenosis.

In one aspect of the invention, peptides are provided which are endothelial cell-binding peptides, i.e., the peptides are specific for endothelial cells. These peptides are also referred to herein as targeting peptides. The peptides of the present invention selectively bind to an endothelial cell surface molecule. A peptide “selectively binds” a cell surface molecule when it interacts with a binding domain of said cell surface molecule with a greater affinity, or is more specific for that binding domain as compared with other binding domains of other cell surface molecules. The phrase “is specific for” refers to the degree of selectivity shown by a peptide with respect to the number and types of interacting molecules with which the peptide interacts and the rates and extent of these reactions, e.g. the degree of selectivity shown by an antibody with respect to the number and types of antigens with which the antibody combines and the rates and the extent of these reactions. The phrase “selectively binds” in the present context also means binding sufficient to be useful in the method of the invention. As is known in the art, useful selective binding, for instance, to a receptor, depends on both the binding affinity and the concentration of ligand achievable in the vicinity of the receptor. Thus, binding affinities lower than that found for any naturally occurring competing ligands may be useful, as long as the cell or tissue to be treated can tolerate concentrations of added ligand sufficient to compete, for instance, for binding to a cell surface receptor.

The term “cell surface molecule” within the meaning of the invention comprises any molecule displayed at the surface membrane of an endothelial cell which will selectively bind to a peptide of the invention. By “cell surface molecule” is meant any site, i.e., a single molecule or a plurality of molecules, present on the surface of a cell with which the peptides of the present invention can interact to bind to the cell.

For the most part, the targeting peptides of the present invention will comprise about 5 to about 50 amino acids, preferably at least about 5 to about 30 amino acids, more preferably at least about 7 to about 20 amino acids most preferably at least 7 to about 10 amino acids. Peptides meeting these parameters are set forth in SEQ ID NOs: 1-37 & 44 (Table 2). It is recognized that consensus sequences may be identified among the peptides that are capable of binding to a target. Such consensus sequences identify key amino acids or patterns of amino acids that are essential for binding. Consensus sequences may be determined by an analysis of peptide patterns that are capable of binding endothelial cells. Once recognized the consensus regions can be used in constructing other peptides for use in endothelial cell targeting. Such consensus sequences may be tested by constructing peptides and determining the effect of the consensus sequence on binding. In this manner, as long as the consensus sequence is present, the peptide will bind the target. In some cases, longer peptides will be useful as such peptides may be more easily bound to the target cell.

Consensus sequences can be determined using standard procedures in the art. One example is using the Pileup program (Wisconsin Package 10.2, Genetic Computer Group (GCG), Madison, Wis.). Analysis of SEQ ID NO:1-37 using Pileup with the default settings revealed a consensus sequence of CXXPTPPXC (SEQ ID NO:44), where X is any amino acid. Thus another embodiment of the invention includes SEQ ID NO:44.

Once peptides have been selected which show an affinity for the target tissue, they may be modified by methods known in the art. Such methods include random mutagenesis, as well as synthesis of the peptides for selected amino acid substitutions. Peptides of various lengths can be constructed and tested for the effect on binding affinity and specificity. In this manner, the binding affinity may be increased or altered. Thus, peptides may be identified which exhibit specific binding to endothelial cells, as well as peptides which exhibit specific binding by the endothelial cells of interest.

The term “targeting peptide” is also intended to include a peptidomimetic of the disclosed peptides. As used herein, the term “peptidomimetic” is used broadly to mean a peptide-like molecule that has the binding activity of the disclosed endothelial cell specific peptides. With respect to the targeting peptides of the invention, peptidomimetics, which include chemically modified peptides, peptide-like molecules containing non-naturally occurring amino acids, peptoids and the like, have the endothelial cells binding activity of the disclosed targeting peptide upon which the peptidomimetic is derived (see, for example, Wolff, 1995).

Targeting peptides of the invention include those of SEQ ID NO: 1 to 37 and 44. One skilled in the art will recognize that all of the sequences have a Cys at positions 1 and 8. Without being bound by theory, the Cysteines at these positions are thought to form a disulfide bond creating a constrained loop. The constrained loop is thought to increase the accessibility and/or exposure of the amino acids at positions 2 to 8. The Cysteines themselves may or may not be involved in the actual binding to the target cell receptor. Therefore targeting peptides of the invention also include a peptide comprising amino, acids 1 to 8 of a sequence selected from the group consisting of SEQ ID NO:1-37 and SEQ ID NO:44; a peptide comprising amino acids 2 to 9 of a sequence selected from the group consisting of SEQ ID NO:1-37 and SEQ ID NO:44; and a peptide comprising amino acids 2 to 8 of a sequence selected from the group consisting of SEQ ID NO:1-37 and SEQ ID NO:44.

Methods for identifying a peptidomimetic are well known in the art and include, for example, the screening of databases that contain libraries of potential peptidomimetics. For example, the Cambridge Structural Database contains a collection of greater than 300,000 compounds that have known crystal structures (Allen et al., 1979). This structural depository is continually updated as new crystal structures are determined and can be screened for compounds having suitable shapes, for example, the same shape as a targeting peptide, as well as potential geometrical and chemical complementarity to a target molecule bound by a targeting peptide. Where no crystal structure of a targeting peptide or a target molecule that binds the targeting peptide is available, a structure can be generated using, for example, the program CONCORD (Rusinko et al., 1989). Another database, the Available Chemicals Directory (Molecular Design Limited, Informations Systems; San Leandro Calif.), contains about 100,000 compounds that are commercially available and also can be searched to identify potential peptidomimetics of a targeting peptide.

Methods for preparing libraries containing diverse populations of various types of molecules such as peptides, peptoids and peptidomimetics are well known in the art and various libraries are commercially available (see, for example, Ecker and Crooke, 1995; Blondelle et al., 1995; Goodman and Ro, 1995; Gordon et al., 1994). Where a molecule is a peptide, protein or fragment thereof, the molecule can be produced in vitro directly or can be expressed from a nucleic acid, which can be produced in vitro. Methods of synthetic peptide and nucleic acid chemistry are well known in the art.

Nucleotide sequences encoding the endothelial-specific peptides are also encompassed. Appropriate nucleotide sequences can be designed on the basis of the genetic code. Thus, the present invention encompasses all nucleotide sequences which would code for the specified peptides. Where necessary, the nucleotide sequences can be used in the construction of fusion proteins or vectors for use in the invention. Such methods are known in the art (see, e.g., WO 00/06195). Additionally the construction of expression cassettes are known as well as promoters, terminators, enhancers, and the like, necessary for expression.

The peptides find use in targeting genes, proteins, pharmaceuticals, radionuclides, liposomes, or other compounds or substances to endothelial cells. In this manner, the peptides can be conjugated to peptides, pharmaceuticals, radionuclides, liposomes or other substances to the target cells. The peptides can be used in any vector systems for delivery of specific nucleotides or compositions to the target cells. By nucleotide is intended gene sequences, DNA, RNA, as well as antisense nucleic acids.

“Polynucleotide”, “nucleotide” and “nucleic acid”, used interchangeably herein, is defined as a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups

As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides In one embodiment, the targeting peptide is genetically incorporated into viral vector particles useful for gene therapy. A number of vector systems are known for the introduction of foreign or native genes into mammalian cells. These include SV40 virus (Okayama et al., 1985); bovine papilloma virus (DiMaio et al., 1982); adenovirus (Morin et al., 1987; Dai et al., 1995; Yang et al., 1996; Tripathy et al., 1996; Quantin et al., 1992; Rosenfeld et al., 1991; Wagner, 1992; Curiel et al., 1992; Curiel, 1991; LeGal LaSalle et al., 1993; Kass-Eisler et al., 1993); adeno-associated virus (Muzyczka, 1994; Xiao et al., 1996); herpes simplex virus (Geller et al., 1988; Huard et al., 1995; U.S. Pat. No. 5,501,979); lentivirus (Douglas et al., 2001; Miyoshi et al., 1999; Garvey et al., 1990; Berkowitz et al., 2001; PCT Publication No. WO 01/144458; U.S. Pat. Nos. 6,277,633 and 5,380,830). The targeting peptides can be used with any mammalian expression vector to target the expression system to the appropriate target endothelial cells. See, for example, Wu et al. (1991); Wu and Wu (1988); Wu et al. (1989); Zenke et al. (1990); and Wagner et al. (1990). Grifman et al. (2001) describes the incorporation of tumor-targeting peptides into recombinant AAV capsids. For descriptive purposes only, this embodiment will be described with reference to an adenoviral vector, a preferred aspect of this embodiment. However, it will be understood that the embodiment is applicable to any of the previously mentioned vector systems and others known in the art.

In a preferred aspect of the first embodiment, the endothelial cell specific peptide, also referred to as targeting peptide or targeting molecule, is genetically incorporated into the capsid of an adenoviral vector particle by modifying the fiber protein to target the adenoviral vector particle. In terms of the loop domains of the fiber knob which can be employed in the context of this embodiment, the crystal structure of the fiber knob has been described (see, e.g., Xia et al., 1994). The knob monomer comprises an eight-stranded antiparallel β-sandwich fold. The overall structure of the fiber knob trimer resembles a three-bladed propeller with certain β-strands of each of the three monomers comprising the faces of the blades. In particular, the following residues of the Ad5 fiber knob appear important in hydrogen bonding in the β-sandwich motif: 400-402, 419-428, 431-440, 454-461, 479-482, 485-486, 516-521, 529-536, 550-557, and 573-578. The remaining residues of the protein (which do not appear to be critical in forming the fiber protein secondary structure) define the exposed loops of the protein knob domain. In particular, residues inclusive of 403-418 comprise the AB loop, residues inclusive of 441-453 comprise the CD loop, residues inclusive of 487-514 comprise the DG loop, residues inclusive of 522-528 comprise the GH loop, residues inclusive of 537-549 comprise the HI loop and residues inclusive of 558-572 comprise the IJ loop.

The term “loop” is meant in the generic sense of defining a span of amino acid residues (i.e., more than one, preferably less than two hundred, and even more preferably, less than thirty) that can be substituted by the nonnative amino acid sequence to comprise a peptide motif that allows for cell targeting. While such loops are defined herein with respect to the Ad5 sequence, the sequence alignment of other fiber species have been described (see, e.g., Xia et al., 1994). For these other species (particularly Ad2, Ad3, Ad7, Ad40 and Ad41 described in Xia et al., 1994), the corresponding loop regions of the knob domains appear to be comparable. Various classes of protein loops are described in Oliva et al. 1997.

Thus, this first embodiment preferably provides a chimeric adenovirus fiber protein comprising a targeting peptide sequence. Preferably, the targeting peptide sequence is constrained by its presence in a loop of the knob of the chimeric fiber protein. In particular, desirably the targeting peptide sequence is inserted into or in place of a protein sequence in a loop of the knob of the chimeric adenoviral fiber protein. Optionally, the fiber protein loop is selected from the group consisting of the AB, CD, DG, GH, and IJ loops, and desirably is the HI loop. Also, preferably, the loop comprises amino acid residues in the fiber knob other than Ad5 residues 400-402, 419-428, 431-440, 454-461, 479-482, 485-486, 516-521, 529-536, 550-557, and 573-578. Desirably, the loop comprises amino acid residues selected from the group consisting of residues 403-418, 441-453, 487-514, 522-528, 537-549, and 558-572. In particular, the targeting peptide sequence present in the loop comprises an amino acid sequence of a targeting peptide described herein. Alternatively, loops can be made in the fiber knobs as described in U.S. Pat. No. 6,057,155.

The targeting peptide sequence is introduced at the level of DNA. Accordingly, the first embodiment also provides an isolated and purified nucleic acid encoding a chimeric adenovirus fiber protein comprising a constrained amino acid sequence of the targeting peptide according to the invention. The means of making such a chimeric fiber protein, particularly the means of introducing the sequence at the level of DNA, is well known in the art (see, for example, Hay et al., 2001; U.S. Pat. Nos. 5,543,328, 5,756,086, and 6,329,190). Briefly, the method comprises introducing a sequence into the sequence encoding the fiber protein so as to insert a new peptide motif into or in place of a protein sequence at the C-terminus of the wild-type fiber protein, or in a loop of a knob of the wild-type fiber protein. Such introduction can result in the insertion of a new peptide binding motif, or creation of a peptide motif (e.g., wherein some of the sequence comprising the motif is already present in the native fiber protein). The method also can be carried out to replace fiber sequences with an amino acid sequence of a targeting peptide according to the present invention.

Generally, this embodiment can be accomplished by cloning the nucleic acid sequence encoding the chimeric fiber protein into a plasmid or some other vector for ease of manipulation of the sequence. Then, a unique restriction site at which further sequences can be added into the fiber protein is identified or inserted into the fiber sequence. A double-stranded synthetic oligonucleotide generally is created from overlapping synthetic single-stranded sense and antisense oligonucleotides such that the double-stranded oligonucleotide incorporates the restriction sites flanking the target sequence and, for instance, can be used to incorporate replacement DNA. The plasmid or other vector is cleaved with the restriction enzyme, and the oligonucleotide sequence having compatible cohesive ends is ligated into the plasmid or other vector to replace the wild-type DNA. Other means of in vitro site-directed mutagenesis such as are known to those skilled in the art, and can be accomplished (in particular, using PCR), for instance, by means of commercially available kits, can also be used to introduce the mutated sequence into the fiber protein coding sequence.

Once the targeting peptide sequence is introduced into the chimeric coat protein, the nucleic acid fragment encoding the sequence can be isolated, e.g., by PCR amplification using 5′ and 3′ primers, preferably ones that terminate in further unique restriction sites. Use of primers in this fashion results in an amplified chimeric fiber-containing fragment that is flanked by the unique restriction sites. The unique restriction sites can be used for further convenient subcloning of the fragment. Other means of generating a chimeric fiber protein also can be employed. These methods are highly familiar to those skilled in the art.

The terms “vector,” “polynucleotide vector,” “polynucleotide vector construct,” “nucleic acid vector construct,” and “vector construct” are also used interchangeably herein to mean any nucleic acid construct for gene transfer, as understood by those skilled in the art.

As used herein, the term “viral vector” is used according to its art-recognized meaning. It refers to a nucleic acid vector construct, which includes at least one element of viral origin and may be packaged into a viral vector particle. The viral vector particles may be utilized for the purpose of transferring DNA, RNA or other nucleic acids into cells either in vitro or in vivo. Viral vectors include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, Sindbis vectors, semliki forest virus vectors, phage vectors, adenoviral vectors, and adeno-associated viral (AAV) vectors. Suitable viral vectors are described in U.S. Pat. Nos. 6,057,155, 5,543,328 and 5,756,086.

The terms “adenovirus vector” and “adenoviral vector” are used interchangeably and are well understood in the art to mean a polynucleotide comprising all or a portion of an adenovirus genome. An adenoviral vector of this invention may be in any of several forms, including, but not limited to, naked DNA, DNA encapsulated in an adenovirus capsid, DNA packaged in another viral or viral-like form (such as herpes simplex, and AAV), DNA encapsulated in liposomes, DNA complexed with polylysine, complexed with synthetic polycationic molecules, conjugated with transferrin, complexed with compounds such as PEG to immunologically “mask” the molecule and/or increase half-life, or conjugated to a non-viral protein.

The terms “viral particle,” “vector particle,” viral vector particle,” “virus,” and “virion” are used interchangeably and are to be understood broadly as meaning infectious viral particles that are formed when, e.g., a viral vector of the invention is transduced into an appropriate cell or cell line for the generation of infectious particles. For purposes of the present invention, these terms preferably refer to adenoviruses, including recombinant adenoviruses formed when an adenoviral vector of the invention is encapsulated in an adenovirus capsid.

As used herein, the terms “adenovirus” and “adenoviral particle” are used to include any and all viruses that may be categorized as an adenovirus, including any adenovirus that infects a human or an animal, including all groups, subgroups, and serotypes. Thus, as used herein, “adenovirus” and “adenovirus particle” refer to the virus itself or derivatives thereof and cover all serotypes and subtypes and both naturally occurring and recombinant forms, except where indicated otherwise. Preferably, such adenoviruses are ones that infect human cells. Such adenoviruses may be wild-type or may be modified in various ways known in the art or as disclosed herein. Such modifications include modifications to the adenovirus genome that is packaged in the particle in order to make an infectious virus. Such modifications include deletions known in the art, such as deletions in one or more of the E1a, E1b, E2a, E2b, E3, or E4 coding regions. Such modifications also include deletions of all of the coding regions of the adenoviral genome. Such adenoviruses are known as “gutless” adenoviruses. The terms also include replication-conditional adenoviruses; that is, viruses that preferentially replicate in certain types of cells or tissues but to a lesser degree or not at all in other types. In a particularly preferred embodiment of the invention, the adenoviral particles replicate in abnormally proliferating tissue, such as solid tumors and other neoplasms. These include the viruses disclosed in U.S. Pat. No. 5,998,205, issued Dec. 7, 1999 to Hallenbeck et al. and U.S. Pat. No. 5,801,029, issued Sep. 1, 1998 to McCormick, the disclosures of both of which are incorporated herein by reference in their entirety. Such viruses are sometimes referred to as cytolytic or cytopathic viruses (or vectors), and, if they have such an effect on neoplastic cells, are referred to as oncolytic viruses (or vectors).

A further embodiment of the invention provides an adenovirus particle comprising a chimeric adenovirus fiber protein comprising a targeting peptide sequence of the invention. The adenoviral vector particle may also include other mutations to the fiber protein. Examples of these mutations include, but are not limited to those described in U.S. provisional application 60/391,967 filed on Jun. 26, 2002, WO 01/92299, U.S. Pat. No. 5,962,311, WO 98/07877 and U.S. Pat. No. 6,153,435. These include, but are not limited to mutations that decrease binding of the viral vector particle to a particular cell type or more than one cell type, enhance the binding of the viral vector particle to a particular cell type or more than one cell type and/or reduce the immune response to the adenoviral vector particle in an animal. In addition, the adenoviral vector particles of the present invention may also contain mutations to other viral capsid proteins. Examples of these mutations include, but are not limited to those described in U.S. Pat. No.5,731,190, U.S. Pat. Nos. 6,127,525, 5,922,315.

The adenoviral vectors of the invention are made by standard techniques known to those skilled in the art. The vectors are transferred into packaging cells by techniques known to those skilled in the art. Packaging cells provide complementing functions to the functions provided by the genes in the adenovirus genome that are to be packaged into the adenovirus particle. The production of such particles requires that the vector be replicated and that those proteins necessary for assembling an infectious virus be produced. The packaging cells are cultured under conditions that permit the production of the desired viral vector particle. The particles are recovered by standard techniques. The preferred packaging cells are those that have been designed to limit homologous recombination that could lead to wild-type adenoviral particles. Such cells are disclosed in U.S. Pat. No. 5,994,128, issued Nov. 30, 1999 to Fallaux, et al., and U.S. Pat. No. 6,033,908, issued Mar. 7, 2000 to Bout, et al. The packaging cell known as PER.C6, which is disclosed in these patents, is particularly preferred.

An especially preferred vector according to this embodiment is an adenoviral vector (i.e., a viral vector of the family Adenoviridae, optimally of the genus Mastadenovirus). Desirably such a vector is an Ad2, Ad5 or Ad35 based vector, although other serotype adenoviral vectors can be employed. Adenoviral stocks that can be employed according to the invention include any adenovirus serotype. Adenovirus serotypes 1 through 47 are currently available from American Type Culture Collection (ATCC, Manassas, Va.), and the invention includes any other serotype of adenovirus available from any source including those serotypes listed in Table 1. The adenoviruses that can be employed according to the invention may be of human or non-human origin. For instance, an adenovirus can be of subgroup A (e.g., serotypes 12, 18, 31), subgroup B (e.g., serotypes 3, 7, 11, 14, 16, 21, 34, 35), subgroup C (e.g., serotypes 1, 2, 5, 6), subgroup D (e.g., serotypes 8, 9, 10, 13, 15, 17, 19, 20, 22-30, 32, 33, 36-39, 42-47), subgroup E (serotype 4), subgroup F (serotype 40, 41), or any other adenoviral serotype.

The adenoviral vector employed for gene transfer can be replication competent. Alternately, the adenoviral vector can comprise genetic material with at least one modification therein, which can render the virus replication deficient. In addition, the adenoviral vector can comprise a genetic material with at least one modification therein, which can render the virus replication conditional, i.e., only capable of replication in specific cells or tissues such as oncolytic adenoviral vectors (e.g. WO 96/17053 and WO 99/25860). The adenoviral vector can further comprise additional sequences and mutations, e.g., some within the fiber protein itself. For instance, a vector according to the invention can comprise a nucleic acid comprising a passenger gene, usually a heterologous gene, preferably a therapeutic gene or a reporter gene.

The means of making the recombinant adenoviral vectors and particles according to the invention are known to those skilled in the art. For instance, a recombinant adenovirus comprising a chimeric fiber protein and a recombinant adenovirus that additionally comprises a passenger gene or genes capable of being expressed in a particular cell can be generated by use of a transfer vector, preferably a viral or plasmid transfer vector, in accordance with the present invention. Such a transfer vector preferably comprises a chimeric adenoviral fiber sequence as previously described. The chimeric fiber protein gene sequence comprises a nonnative (i.e., non-wild-type) sequence in place of the native sequence, which has been deleted, or in addition to the native sequence.

TABLE 1
Examples Of Human And Animal Adenoviruses Including The American Type Culture Collection
Catalog # For A Representative Virus Of The Respective Classification

| | |
|---|---|
| Avian adenovirus Type 2 (GAL) ATCC VR-280 | Adenovirus Type 38 ATCC VR-988 |
| Adenovirus Type 2 Antiserum: ATCC VR-1079 | Adenovirus Type 46 ATCC VR-1308 |
| Adenovirus Type 21 ATCC VR-1099 | Simian adenovirus ATCC VR-541 |
| SA18 (Simian adenovirus 18) ATCC VR-943 | SA7 (Simian adenovirus 16) ATCC VR-941 |
| SA17 (Simian adenovirus 17) ATCC VR-942 | Frog adenovirus (FAV-1) ATCC VR-896 |
| Adenovirus Type 47 ATCC VR-1309 | Adenovirus type 48 (candidate) ATCC VR-1406 |
| Adenovirus Type 44 ATCC VR-1306 | Adenovirus Type 42 ATCC VR-1304 |
| Avian adenovirus Type 4 ATCC VR-829 | Adenovirus Type 49 (candidate) ATCC VR-1407 |
| Avian adenovirus Type 5 ATCC VR-830 | Adenovirus Type 43 ATCC VR-1305 |
| Avian adenovirus Type 7 ATCC VR-832 | Avian adenovirus Type 6 ATCC VR-831 |
| Avian adenovirus Type 8 ATCC VR-833 | Avian adenovirus Type 3 |
| Avian adenovirus Type 9 ATCC VR-834 | Bovine adenovirus Type 3 ATCC VR-639 |
| Avian adenovirus Type 10 ATCC VR-835 | Adenovirus Type 1 ATCC VR-1078 |
| Avian adenovirus Type 2 ATCC VR-827 | Adenovirus Type 21 ATCC VR-256 |
| Adenovirus Type 45 ATCC VR-1307 | Adenovirus Type 18 ATCC VR-1095 |
| Bovine adenovirus Type 6 ATCC VR-642 | Baboon adenovirus ATCC VR-275 |
| Canine adenovirus ATCC VR-800 | Adenovirus Type 10 ATCC VR-11 |
| Bovine adenovirus Type 5 ATCC VR-641 | Adenovirus Type 33 ATCC VR-626 |
| Adenovirus Type 36 ATCC VR-913 | Adenovirus Type 34 ATCC VR-716 |
| Ovine adenovirus type 5 ATCC VR-1343 | Adenovirus Type 15 ATCC VR-16 |
| Adenovirus Type 29 ATCC VR-272 | Adenovirus Type 22 ATCC VR-257 |
| Swine adenovirus ATCC VR-359 | Adenovirus Type 24 ATCC VR-259 |
| Bovine adenovirus Type 4 ATCC VR-640 | Adenovirus Type 17 ATCC VR-1094 |
| Bovine adenovirus Type 8 ATCC VR-769 | Adenovirus Type 4 ATCC VR-1081 |
| Bovine adenovirus Type 7 ATCC VR-768 | Adenovirus Type 16 ATCC VR-17 |
| Adenovirus Type 4 ATCC VR-4 | Adenovirus Type 17 ATCC VR-18 |
| Peromyscus adenovirus ATCC VR-528 | Adenovirus Type 16 ATCC VR-1093 |
| Adenovirus Type 15 ATCC VR-661 | Infectious canine hepatitis (Rubarth's disease) |
| Adenovirus Type 20 ATCC VR-662 | Bovine adenovirus Type 2 ATCC VR-314 |
| Chimpanzee adenovirus ATCC VR-593 | SV-30 ATCC VR-203 |
| Adenovirus Type 31 ATCC VR-357 | Adenovirus Type 32 ATCC VR-625 |
| Adenovirus Type 25 ATCC VR-223 | Adenovirus Type 20 ATCC VR-255 |
| Chimpanzee adenovirus ATCC VR-592 | Adenovirus Type 13 ATCC VR-14 |
| Chimpanzee adenovirus ATCC VR-591 | Adenovirus Type 14 ATCC VR-1091 |
| Adenovirus Type 26 ATCC VR-224 | Adenovirus Type 18 ATCC VR-19 |
| Adenovirus Type 19 ATCC VR-254 | SV-39 ATCC VR-353 |
| Adenovirus Type 23 ATCC VR-258 | Adenovirus Type 11 ATCC VR-849 |
| Adenovirus Type 28 ATCC VR-226 | Duck adenovirus(Egg drop syndrome)VR-921 |
| Adenovirus Type 6 ATCC VR-6 | Adenovirus Type 1 ATCC VR-1 |
| Adenovirus Type 6 ATCC VR-1083 | Chimpanzee adenovirus ATCC VR-594 |
| Ovine adenovirus Type 6 ATCC VR-1340 | Adenovirus Type 15 ATCC VR-1092 |
| Adenovirus Type 3 ATCC VR-847 | Adenovirus Type 13 ATCC VR-1090 |
| Adenovirus Type 7 ATCC VR-7 | Adenovirus Type 19 ATCC VR-1096 |
| Adenovirus Type 39 ATCC VR-932 | SV-36 ATCC VR-208 |
| Adenovirus Type 3 ATCC VR-3 | SV-38 ATCC VR-355 |
| Bovine adenovirus Type 1 ATCC VR-313 | SV-25 (M8) ATCC VR-201 |
| Adenovirus Type 14 ATCC VR-15 | SV-15 (M4) ATCC VR-197 |
| Adenovirus Type 8 ATCC VR-1368 | Adenovirus Type 22 ATCC VR-1100 |
| SV-31 ATCC VR-204 | SV-23 (M2) ATCC VR-200 |
| Adenovirus Type 9 ATCC VR-1086 | Adenovirus Type 11 ATCC VR-12 |
| Mouse adenovirus ATCC VR-550 | Adenovirus Type 24 ATCC VR-1102 |
| Adenovirus Type 9 ATCC VR-10 | Avian adenovirus Type 1 |
| Adenovirus Type 41 ATCC VR-930 | SV-11 (M5) ATCC VR-196 |
| C1 ATCC VR-20 | Adenovirus Type 5 ATCC VR-5 |
| Adenovirus Type 40 ATCC VR-931 | Adenovirus Type 23 ATCC VR-1101 |
| Adenovirus Type 37 ATCC VR-929 | SV-27 (M9) ATCC VR-202 |
| Marble spleen disease virus | SV-1 (M1) ATCC VR-195 |
| Adenovirus Type 35 ATCC VR-718 | SV-17 (M6) ATCC VR-198 |
| SV-32 (M3) ATCC VR-205 | Adenovirus Type 29 ATCC VR-1107 |
| Adenovirus Type 28 ATCC VR-1106 | Adenovirus Type 2 ATCC VR-846 |
| Adenovirus Type 10 ATCC VR-1087 | SV-34 ATCC VR-207 |
| Adenovirus Type 20 ATCC VR-1097 | SV-20 (M7) ATCC VR |
| Adenovirus Type 21 ATCC VR-1098 | SV-37 ATCC VR-209 |
| Adenovirus Type 25 ATCC VR-1103 | SV-33 (M10) ATCC VR-206 |
| Adenovirus Type 26 ATCC VR-1104 | Adenovirus Type 30 ATCC VR-273 |
| Adenovirus Type 31 ATCC VR-1109 | Adenovirus Type 27 ATCC VR-1105 |
| | Adenovirus Type 12 ATCC VR-863 |
| | Adenovirus Type 7a ATCC VR-8 |

A vector according to the invention further can comprise, either within, in place of, or outside of the coding sequence of a fiber protein additional sequences that impact upon the ability of the fiber protein to trimerize, or comprise a protease recognition sequence. A sequence that impacts upon the ability to trimerize is one or more sequences that enable fiber trimerization.

In terms of the production of vectors and transfer vectors according to this embodiment, transfer vectors are constructed using standard molecular and genetic techniques such as are known to those skilled in the art. Virions or virus particles are produced using viral vectors in the appropriate cell lines. Similarly, the adenoviral fiber chimera-containing particles are produced in standard cell lines, e.g., those currently used for adenoviral vectors. An adenovirus lacking fiber can be produced as described in PCT Publication WO 00/42208.

The present embodiment provides a chimeric fiber protein that is able to bind to endothelial cells and mediate transduction of endothelial cells with high efficiency, as well as vectors and transfer vectors comprising the same.

The vectors and transfer vectors of the present invention can be employed to contact cells either in vitro or in vivo. The method is not dependent on any particular means of introduction and is not to be so construed. Means of introduction are well known to those skilled in the art. Accordingly, the complexes of this embodiment may be administered in vivo to a host. The host may be an animal host, including mammalian hosts, primate hosts and human hosts. Thus, the complex is useful as a medicament and useful for the preparation of a medicament for the treatment of a disease in a mammal including a human.

Thus, this embodiment also provides a method of targeting an adenoviral particle to a cell which expresses a cell surface molecule comprising the steps of contacting said adenoviral particle having a fiber protein modified to contain a targeting peptide suitable to target said cell surface molecule and contacting said cell with said particle.

This embodiment further provides a method of delivering an adenoviral particle selectively to a cell which expresses a cell surface molecule comprising the steps of contacting an adenoviral particle which comprises said adenoviral vector with a fiber protein modified to contain a targeting peptide to target said cell surface molecule, and contacting said cell with said adenoviral particle.

Furthermore, this embodiment also provides a method of delivering a heterologous gene selectively to a cell which expresses a cell surface molecule comprising the steps of contacting an adenoviral particle which comprises said heterologous gene and a fiber protein modified to contain a targeting peptide suitable for targeting said cell surface molecule and contacting said cell with said adenoviral particle.

The complex may be administered in an amount effective to provide a therapeutic effect in a host. In one aspect, the viral particle may be administered in an amount of from 1 viral particle to about 10¹⁴ viral particles, preferably from about 10⁶ viral particles to about 10¹³ viral particles. The host may be a human or non-human animal host. Preferably, the complex particles are administered systemically, such as, for example, by intravenous administration (such as, for example, portal vein injection or peripheral vein injection), intramuscular administration, intraperitoneal administration, intraocular administration, or intranasal administration. The complex particles may be administered in combination with a pharmaceutically acceptable carrier suitable for administration to a patient. The carrier may be a liquid carrier (for example, a saline solution), or a solid carrier, such as, for example, microcarrier beads. The complex particles, travel directly to the desired cells or tissues upon the in vivo administration of such complex particles to a host. The targeted viral particles then infect the desired cell or tissues.

Standard techniques for the construction of the vectors of the present invention are well known to those of ordinary skill in the art and can be found in such references as Sambrook et al. (1989) and Sambrook and Russel (2001). A variety of strategies are available for ligating fragments of DNA, the choice of which depends on the nature of the termini of the DNA fragments and which choices can be readily made by those of skill in the art.

Where the peptides of the invention are targeting a gene for expression, the gene to be expressed will be provided in an expression cassette with the appropriate regulatory elements necessary for expression of the gene in the targeted cell type. Such regulatory elements are well known in the art and include promoters, terminators, enhancers and the like.

In a second embodiment, the peptide is genetically incorporated into the soluble receptor of, preferably, adenoviral vector particles and thus detarget and retarget the particles (WO 02/29072, which is incorporated herein by reference). In this embodiment the endothelial cell specific peptides are used as a targeting ligand domain to provide a targeting strategy that employs a soluble adenoviral receptor domain, such as the extracellular domain of CAR (sCAR). A targeting ligand domain is appended to the soluble adenoviral receptor domain, and then the conjugate is added to an adenoviral particle. The conjugate binds to the fiber knob of the adenoviral particle to form a complex and thereby redirects the particle to a different cell surface molecule. It is preferred to provide trimerization of the soluble adenoviral receptor domain to enhance the binding of such a targeting molecule to the adenoviral particle. The adenoviral particles complexed with targeting molecules which include a trimerization domain and a targeting ligand domain efficiently transduce cells in vitro and in vivo. This approach of re-targeting an adenoviral particle does not require the generation of adenoviral particles with modified fiber or other capsid proteins. Adenoviral particles can be prepared and grown to high titer using normal protocols and standard cell lines. The addition of a soluble adenoviral receptor domain, such as sCAR, fused to a targeting ligand domain inhibits the normal tropism of the adenoviral vector particle and simultaneously redirects it to the target of choice.

A soluble adenoviral receptor domain may be a fragment or a chemically modified fragment, or even the entire part of an adenoviral receptor molecule which retains binding specificity for an adenoviral fiber protein and may be dissolved in aqueous solution under physiological conditions. Preferably, the soluble adenoviral receptor domains are isolated extracellular domains of adenoviral receptor domains. In a preferred embodiment the soluble adenoviral receptor domain is sCAR. The CAR cDNA sequence is known in the art and is published under GenBank accession number Y07593. In one embodiment of the present invention sCAR comprises at least base pairs 60 to 487 of the published CAR cDNA sequence, extending from the ATG codon through the first Ig-like domain, termed the D1 domain. A preferred sCAR-sequence of this invention includes base pairs 54 to 767 of the CAR sequence.

The trimerization domain of the targeting molecule may be a heterologous trimerization domain with respect to the soluble adenoviral receptor domain, i.e. it comprises a nonnative amino acid sequence with respect to the soluble adenoviral receptor domain. “Nonnative amino acid sequence” encompasses any amino acid sequence that is not found in the same position in the soluble adenoviral receptor domain and which is introduced into the soluble adenoviral receptor domain, for example at the level of gene expression. Nonnative amino acid sequences include for example an amino acid sequence derived from a leucine zipper molecule, such as a yeast leucine zipper molecule. In one aspect the nonnative amino acid sequence is a variant of the yeast leucine zipper molecule in which certain key leucine residues are mutated to isoleucine residues, such as in Harbury et al. (1993). The trimerization domain confers upon the soluble adenoviral receptor domain the ability to form a trimer, in particular a homotrimer, directly or indirectly. Indirect homotrimerization may for example be achieved via a bispecific or multispecific binding agent, such as an antibody or fragment thereof, which interacts with the trimerization or other domain in the soluble receptor.

The trimerization domain may be localized downstream of the C-terminus of the soluble adenoviral receptor domain. The trimerization domain may also be introduced into the sequence of the soluble adenoviral receptor domain. If the trimerization domain is introduced into the sequence of the soluble adenoviral receptor domain, it is preferably introduced into the carboxy-terminal end. The trimerization domain may include any of those disclosed in WO 02/29072, which is incorporated herein by reference.

It will be readily appreciated by the person skilled in the art that important criteria for selecting a suitable trimerization domain in a particular setting are, first, its “strength” and, second, its “size”. The strength of the trimerization domain may be quantified as the stability of the trimeric molecule formed under defined conditions, as measurable for example in its association / dissociation kinetics. The size of the trimerization domain (in particular the total number of amino acids of the trimerization domain) may be a criterion of choice in the construction of a particular targeting molecule because the trimerization domain should be small enough to be incorporated into the soluble adenoviral receptor domain without disrupting its binding function.

In yet another aspect, the targeting molecule further comprises a linker element which is localized between the carboxy-terminal end of the adenoviral receptor domain and the trimerization domain. The linker element may preferably be a peptide linker. As used herein, the term “peptide linker” refers to a short peptide sequence serving as a spacer e.g. between the carboxy-terminal end of the adenoviral receptor domain and the trimerization domain. Such a sequence desirably is incorporated into the protein to ensure that the trimerization domains are not sterically hindered by the soluble adenoviral receptor domains and are capable to interact and efficiently form homotrimers. A linker sequence can be of any suitable length, preferably from about 3 to about 30 amino acids, and comprises any amino acids, for instance, a mixture of glycine and serine residues. Optimally, the linker sequence does not interfere with the functioning of the soluble adenoviral receptor domain. In a preferred aspect the linker element consists of alternating glycine and serine residues.

The targeting molecule may also be assembled or combined, wholly or partly, by non-covalently binding each domain.

This embodiment further provides a complex comprising an adenoviral particle and the targeting molecule. A “complex” of the adenoviral particle and the targeting molecule is any interaction, e.g., covalent or noncovalent, between the adenoviral particle and the targeting molecule. Preferably, it is a noncovalent interaction. Complex formation occurs when the adenoviral particle and the targeting molecule are contacted. Such “contacting” can be done by any means known to those skilled in the art and described herein, by which the mutual tangency of the adenovirus and targeting molecule can be effected. For instance, contacting of the adenoviral particle and the targeting molecule can be done by mixing these elements in a small volume of the same solution. For example, the adenoviral particle and the targeting molecule can be allowed to associate for 30 minutes at 37° C. in a suitable solution. Optionally, the adenoviral particle and the targeting molecule further can be covalently joined, e.g., by chemical means known to those skilled in the art, or other means, or, preferably, can be linked by means of noncovalent interactions (e.g., ionic bonds, hydrogen bonds, van der Waals forces, and/or nonpolar interactions). Preferably, the complex of the adenovirus and the targeting molecule is formed prior to the contacting of the cell. This period of time may be about as long as the maximum length of time a complex of an adenovirus and a targeting molecule can be stably maintained in a useable form, for instance, lyophilized, or in the presence of cryoprotective agents at −80° C.

This embodiment also provides a polynucleotide encoding the amino acid sequence of the targeting molecule of the invention. Also provided is a polynucleotide that is a variant of such a polynucleotide and encodes a corresponding functional variant of the amino acid sequence of the targeting molecule. A functional variant may differ in amino acid sequence by one or more substitutions, additions, deletions, truncations which may be present in any combination, but would retain the same biological function as the referee targeting molecule, such as described in WO 02/29072, which is incorporated herein by reference.

“Biological function” within the meaning of this application is to be understood in a broad sense. It includes, but is not limited to, the particular functions of the elements of the targeting molecule disclosed in this application, the element being the soluble adenoviral receptor domain, the trimerization domain and the targeting ligand domain. Thus, biological functions are not only those which a polypeptide displays in its physiological context, i.e. as part of a living organism or cell, but includes functions which it may perform in a non-physiological setting, e.g. in vitro. For example, a biological function of the soluble adenoviral receptor domain within the meaning of this application is the ability to bind to the fiber protein of an adenoviral particle of the invention either in vitro or in vivo. A biological function of the trimerization domain within the meaning of this application is the ability to trimerize the targeting molecule of the invention in vitro and to maintain the trimeric state in vivo. A biological function of the targeting ligand domain within the meaning of this application is the ability to bind to a corresponding cell surface molecule as defined in this application in vitro or in vivo. Assays to assess the required properties, for example the binding properties of the proteins to specific ligands are well-known in the art.

The means of making such a targeting molecule, in particular the means of introducing the sequence of the trimerization domain into the sequence of the soluble adenoviral receptor domain or at the 3′ end of the soluble adenoviral receptor domain at the level of DNA, is well known in the art, and is further described in WO 02/29072, which is incorporated herein by reference. Briefly, the method comprises introducing a sequence of the chosen trimerization domain into the sequence encoding the chosen soluble adenoviral receptor domain so as to insert a new peptide motif into or in place of a protein sequence of the wild-type soluble adenoviral receptor domain. Such introduction can result in the insertion of a new peptide binding motif, or creation of a peptide motif, e.g. wherein some of the sequence comprising the motif is already present in the wild-type soluble adenoviral receptor domain. The method also can be carried out to replace sequences of the soluble adenoviral receptor domain with a nonnative amino acid sequence according to the invention. Generally, this can be accomplished by cloning the nucleic acid sequence encoding the soluble adenoviral receptor domain into a plasmid or some other vector for ease of manipulation of the sequence. Then, a unique restriction site at which further sequences can be added is identified or inserted into the sequence of the plasmid including the sequence of the soluble adenoviral receptor domain. A double-stranded synthetic oligonucleotide generally is created from overlapping synthetic single-stranded sense and antisense oligonucleotides such that the double-stranded oligonucleotide incorporates the restriction sites flanking the target sequence and, for instance, can be used to incorporate replacement DNA. The plasmid or other vector is cleaved with the restriction enzyme, and the oligonucleotide sequence having compatible cohesive ends is ligated into the plasmid or other vector to replace the wild-type DNA. Other means that are known to those skilled in the art, in particular using PCR techniques, can also be used to introduce the sequence of the trimerization domain into the soluble adenoviral receptor domain coding sequence.

This second embodiment of the invention further provides an expression vector comprising a polynucleotide encoding the nucleic acid sequence of the targeting molecule, or comprising at least two polynucleotides encoding for a ligand molecule and a soluble adenoviral receptor molecule optionally further comprising in sequence a trimerization domain. A suitable expression vector is any vector that includes all necessary genetic elements for the expression of the inserted DNA sequence when propagated in a suitable host cell. Numerous suitable expression vectors are known to the person skilled in the art and are commercially available.

This embodiment provides a complex comprising an adenoviral particle and the targeting molecule.

This embodiment also provides a method of targeting an adenoviral particle to a cell which expresses a cell surface molecule comprising the steps of contacting said adenoviral particle with a targeting molecule which comprises a soluble adenoviral receptor domain, a trimerization domain and a targeting peptide domain, obtaining a complex suitable to target said cell surface molecule and contacting said cell with said complex.

This embodiment further provides a method of delivering an adenoviral vector selectively to a cell which expresses a cell surface molecule comprising the steps of contacting an adenoviral particle which comprises said adenoviral vector with a targeting molecule which comprises a soluble adenoviral receptor domain, a trimerization domain and a targeting peptide domain, obtaining a complex suitable to target said cell surface molecule, and contacting said cell with said complex.

Furthermore, this embodiment also provides a method of delivering a heterologous gene selectively to a cell which expresses a cell surface molecule comprising the steps of contacting an adenoviral particle which comprises said heterologous gene with a targeting molecule which comprises a soluble adenoviral receptor domain, a trimerization domain and a targeting peptide domain, obtaining a complex which is suitable for targeting said cell surface molecule and contacting said cell with said complex.

Accordingly, the complexes of this embodiment may be administered ill vivo to a host. The host may be an animal host, including mammalian hosts, primate hosts and human hosts. Thus, the complex is useful as a medicament and useful for the preparation of a medicament for the treatment of a disease in a mammal including a human.

The complexes of this embodiment may also be administered in vitro to cells. This may be done in the context of ex vivo gene therapy. Also, these complexes can be used as a general method of gene transfer.

In a third embodiment, the targeting peptide is genetically incorporated into a retroviral or lentiviral vector particle (PCT Publication No. WO 98/44938, incorporated herein by reference; Gollan and Green, 2002; U.S. Pat. Nos. 6,004,798 and 5,985,655; and PCT Publication Nos. WO 98/51700 and WO 94/11524). Retrovirus- and lentivirus-based vectors are well known in the art (Curran et al., 1982; Gazit et al., 1986; Miller, 1992; Kavanaugh et al., 1994; Smith et al., 1990; PCT Publication Nos. WO 98/44938; WO 01/44458; and U.S. patent application Ser. No. 60/353,177). The targeting peptide is preferably incorporated into a viral surface protein, such as a viral envelope polypeptide, to provide retroviral particles targeted to endothelial cells. The targeting peptide may be placed in any region of any viral surface protein which will allow specific targeting by the targeting peptide. The targeting peptide, in one aspect, may be placed between two consecutive amino acid residues of a viral surface protein. Alternatively, amino acid residues of a viral surface protein are removed and replaced with the targeting peptide.

In one aspect, the receptor binding region of the retroviral envelope is modified to include the targeting peptide. For example, the targeting peptide may be inserted between amino acid residues 6 and 7 or between amino acid residues 18 and 19 of a receptor binding region of an ecotropic retroviral envelope, such as described in PCT Publication No. WO 98/44938. As an alternative to modifying the receptor binding region, or in addition to the modified receptor binding region, the retroviral particles may have modifications in other regions of the envelope protein such that other regions of the envelope may include the targeting peptide, such as, for example, the secretory signal or “leader” sequence, the hinge region, or the body portion. Such modifications may include deletions or substitutions of amino acid residues in the retroviral envelope wherein amino acid residues from regions other than the receptor binding region of the envelope are removed and replaced with the targeting peptide, or the targeting polypeptide is placed between consecutively numbered amino acid residues of regions other than the receptor binding region of the viral envelope.

In another alternative aspect, the retroviral envelope, prior to modification thereof to include the targeting peptide which binds to the extracellular matrix component, may be an envelope which includes regions of different tropisms. For example, the retroviral envelope may be a Moloney Murine Leukemia Virus envelope which includes a gp70 protein having an ecotropic portion and an amphotropic and/or xenotropic portion.

In another aspect, the retroviral vector particle includes a first retroviral envelope and a second retroviral envelope. Each of the first retroviral envelope and the second retroviral envelope includes a surface protein. The surface protein includes (i) a receptor binding region; (ii) a hypervariable polyproline, or “hinge” region; and (iii) a body portion. The receptor binding region, hypervariable polyproline region, and body portion are retained in the first retroviral envelope, which in general, is free of non-retroviral peptides. In the second retroviral envelope, a targeting peptide including a binding region which binds to an extracellular matrix component, as hereinabove described, is inserted between two contiguous amino acid residues of the surface protein.

The first retroviral envelope may be an amphotropic envelope, an ecotropic envelope, or a xenotropic envelope. Alternatively, the first retroviral envelope may include regions of different tropisms. For example, in one embodiment, the first retroviral envelope may include a surface protein which includes (i) an ecotropic receptor binding region; (ii) an amphotropic hypervariable polyproline region; and (iii) an ecotropic body portion. The second retroviral envelope may be an amphotropic envelope, an ecotropic envelope, or a xenotropic envelope, or an envelope having different tropisms, as hereinabove described.

In addition to the binding region, the targeting peptide may further include linker sequences of one or more amino acid residues, placed at-the N-terminal and/or C-terminal of the binding region, whereby such linkers affect rotational flexibility and/or steric hindrance of the modified envelope polypeptide. Preferably, the linker increases rotational flexibility and/or minimizes steric hinderance.

In accordance with one aspect of this third embodiment, there is provided a modified polynucleotide encoding a modified viral surface protein for targeting a vector to endothelial cells. Such polynucleotide includes a polynucleotide encoding a targeting peptide including a binding region which binds to endothelial cells. The vector and modified viral surface protein may be selected from those well known in the art and as described herein. The polynucleotide is prepared using conventional techniques, such as those well known in the art, those described herein and those described in WO 98/44938, WO 01/44458, and U.S. patent application No. 60/353,177, entitled “Recombinant Bovine Immunodeficiency Virus Based Gene Transfer System.” For example, a first expression plasmid may be constructed which includes a polynucleotide encoding the unmodified envelope. The plasmid then is engineered such that a polynucleotide encoding the targeting peptide is inserted between two codons encoding consecutively numbered amino acid residues of the unmodified envelope, or is engineered such that a polynucleotide encoding a portion of the unmodified envelope is removed, whereby such portion may be replaced with a polynucleotide encoding the targeting peptide. The polynucleotide encoding the targeting peptide may be contained in a second expression plasmid or may exist as a naked polynucleotide sequence. The polynucleotide encoding the targeting peptide or the plasmid containing such polynucleotide is cut at appropriate restriction enzyme sites and cloned into the first expression plasmid which also has been cut at appropriate restriction enzyme sites. The resulting expression plasmid thus includes a polynucleotide encoding the modified envelope protein. Such polynucleotide then may be cloned out of the expression vector, and into a retroviral vector. The resulting retroviral vector, which includes the polynucleotide encoding the modified envelope protein, and which also may include a polynucleotide encoding a heterologous protein or peptide, is transfected into an appropriate packaging cell line to form a producer cell line for generating retroviral vector particles including the modified envelope protein.

Alternatively, a naked polynucleotide sequence encoding the modified envelope protein is transfected into a “pre-packaging” cell line including nucleic acid sequences encoding the gag and pol proteins, thereby forming a packaging cell line, or is transfected into a packaging cell line including nucleic acid sequences encoding the gag, pol, and wild-type (i.e., unmodified) env proteins, thereby forming a packaging cell line including nucleic acid sequences encoding wild-type env protein and the modified envelope protein. Such packaging cells then may be transfected with a retroviral vector, which may include a nucleic acid sequence encoding a heterologous protein or peptide, thereby forming a producer cell line for generating retroviral vector particles including the modified envelope protein. Such a polynucleotide thus may be contained in the above-mentioned retroviral vector particle, or in a producer cell for generating the above-mentioned retroviral vector particle.

In a further aspect of this third embodiment, the vector particle having a modified envelope in accordance with the invention includes a polynucleotide encoding a heterologous polypeptide which is to be expressed in a desired cell. The heterologous polypeptide may, in one embodiment, be a therapeutic agent. The term “therapeutic” is used in a generic sense and includes treating agents, prophylactic agents, and replacement agents. The polynucleotides encoding the modified envelope polypeptide and the therapeutic agent may be placed into appropriate vectors by genetic engineering techniques known to those skilled in the art.

This embodiment also provides a method of targeting a retroviral particle to a cell which expresses a cell surface molecule comprising the steps of contacting said retroviral particle having a viral surface protein modified to contain a targeting peptide suitable to target said cell surface molecule and contacting said cell with said particle.

This embodiment further provides a method of delivering a retroviral particle selectively to a cell which expresses a cell surface molecule comprising the steps of contacting a retroviral particle which comprises said retroviral vector with a viral surface protein modified to contain a targeting peptide to target said cell surface molecule, and contacting said cell with said retroviral particle.

Furthermore, this embodiment also provides a method of delivering a heterologous gene selectively to a cell which expresses a cell surface molecule comprising the steps of contacting a retroviral particle which comprises said heterologous gene and a viral surface protein modified to contain a targeting peptide suitable for targeting said cell surface molecule and contacting said cell with said retroviral particle.

Accordingly, the complexes of this embodiment may be administered in vivo to a host. The host may be an animal host, including mammalian hosts, primate hosts and human hosts. Thus, the complex is useful as a medicament and useful for the preparation of a medicament for the treatment of a disease in a mammal including a human.

In a fourth embodiment, the targeting peptide is incorporated into protein or peptide therapeutics, such as growth factors and cytokines (WO 00/06195; Curnis et al., 2002). For descriptive purposes only, this embodiment will be described with reference to growth factors as the therapeutic proteins or peptides. However, it will be understood that this embodiment is also applicable to other protein or peptide therapeutics.

Thus, in one aspect of this embodiment, the invention provides fusion polypeptides comprising a targeting peptide and a growth factor or a growth factor fragment. These fusion proteins are capable of binding to endothelial cells. A “fusion protein” is a polypeptide containing portions of amino acid sequence derived from two or more different proteins, or two or more regions of the same protein that are not normally contiguous. A fragment refers to a portion of a protein, e.g., a growth factor, which exhibits growth factor activity, i.e., the growth factor fragment retains substantially the same biological activity as the full length growth factor. In addition, fragments can have the same or substantially the same amino acid sequence as the naturally occurring protein. “Substantially the same” means that an amino acid sequence is largely, but not entirely, the same, but retains a functional activity of the sequence to which it is related. In general two amino acid sequences are substantially the same” or “substantially homologous” if they are at least 85% identical, or if there are conservative variations in the sequence. A computer program, such as the BLAST program (Altschul et al., 1990) can be used to compare sequence identity, and the ALOM (Klein et al., 1985) can be used in analyzing amino acid sequences for potential peripheral and membrane-spanning regions.

As used herein, “growth factor” refers to any peptide factor which transmits signals between cells. Thus, the term “growth factor” includes cytokines, lymphokines, monokines, interferons, colony stimulating factors and chemokines. Examples of growth factors include, but are not limited to, angiopoeitin-1, epidermal growth factor (EGF) , hepatocyte growth factor (HGF), tumor necrosis factor (TNF-alpha), platelet derived endothelial cell growth factor (PD-ECGF), platelet derived growth factor (PDGF), insulin-like growth factor (IGF), interleukin-8, growth hormone, angiopoietin, vascular endothelial growth factor (VEGF), acidic and basic fibroblast growth factors (FGFs), transforming growth factor alpha (TGF-a), CYR 61 and platelet-derived growth factor (PDGF). In addition, see McKay and Leigh (1993) for additional growth factors.

In an additional aspect of the this fourth embodiment, the invention provides isolated nucleic acid sequences which encode a fusion polypeptide containing a targeting peptide linked to a growth factor, or a functional fragment thereof. By “isolated nucleic acid sequence” is meant a polynucleotide that is not immediately contiguous with both the sequences with which it is immediately contiguous (one on the 5′ end and one on the 3′ end) in the naturally occurring genome of the organism from which it is derived. The term therefore includes, for example, a recombinant DNA (a) which is incorporated into (i) a vector, (ii) an autonomously replicating plasmid or virus; or (iii) the genomic DNA of a prokaryote or eukaryote, or (b) which exists as a separate molecule (e.g., a cDNA) independent of other sequences. The nucleotides of the invention can be ribonucleotides, deoxyribonucleotides, or modified forms of either nucleotide. The term includes single and double stranded forms of DNA.

Nucleic acid sequences which encode a targeting peptide linked to a growth factor, or functional fragment thereof, can be operatively linked to expression control sequences. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences regulate and control the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences are well known in the art, and any appropriate sequence can be used.

In a further aspect of this embodiment, the present invention provides vectors in which the nucleic acid sequences encoding the fusion polypeptide of this embodiment have been inserted. Vectors include cloning vectors, helper vectors, expression vectors and other vectors well known in the art. The term “expression vector” refers to a vector known in the art that has been manipulated by insertion or incorporation of the nucleic acid sequences encoding the fusion peptides of the invention. Such vectors are well known in the art. Transformed cells containing the vectors are also provided by this embodiment. Suitable cells include prokaryotic cells and eukaryotic cells, all of which are well known in the art. Finally, this embodiment provides for the production of the fusion proteins by expression of the nucleic acid encoding fusion proteins in transformed cells in accordance with techniques well known in the art. The fusion proteins are isolated and purified by conventional techniques.

Pharmaceutical compositions of the fusion proteins are prepared by conventional methods. The pharmaceutical composition or the fusion protein is administered in accordance with procedures well known in the art. Alternatively, a nucleic acid encoding the fusion protein is administered in accordance with conventional procedures.

In a fifth embodiment, the peptide is incorporated into bi-functional peptides, e.g., the bi-functional peptide containing the peptide of the present invention as a targeting domain (the first functional peptide) and also containing a therapeutic functional domain (the second functional domain), such as a pro-apoptotic domain (Ellerby et al., 1999; Arap et al., 2002), a toxin domain (such as ricin) and the like. In the description which follows, an antiangiogenic peptide is used as the second functional domain. However, it is understood that this embodiment is not limited to an antiangiogenic peptide as the second functional domain.

Additional components can be included as part of the bifunctional peptide, if desired. For example, in some cases, it can be desirable to utilize an oligopeptide spacer between a targeting peptide and the antiangiogenic peptide to impart, for example, flexibility to the bifunctional peptide. Such spacers are well known in the art, as described, for example, in Fitzpatrick and Garnett (1995), and may include, for example, a glycinylglycine linker, alaninylalanine linker or other linker incorporating glycine, alanine or other amino acids.

A bifunctional peptide of the fifth embodiment can readily be synthesized in required quantities using routine methods of solid state peptide synthesis. Alternatively, fusion proteins of the two peptides and any optional additional components can readily be prepared as described above. In addition, the two peptides can be separately synthesized and/or isolated and then linked together. Several methods can be used to link a second functional peptide to a targeting peptide are known in the art, depending on the particular chemical characteristics of the peptides. For example, methods of linking haptens to carrier proteins as used routinely in the field of applied immunology (see, for example, Harlow and Lane, 1988; Hermanson, 1996).

A premade second functional peptide (such as an antiangiogenic peptide) also can be conjugated to a targeting peptide using, for example, carbodiimide conjugation (Bauminger and Wilchek, 1980). Carbodiimides comprise a group of compounds that have the general formula RN═C=NR′, where R and R′ can be aliphatic or aromatic, and are used for synthesis of peptide bonds. The preparative procedure is simple, relatively fast, and is carried out under mild conditions. Carbodiimide compounds attack carboxylic groups to change them into reactive sites for free amino groups. Carbodiimide conjugation has been used to conjugate a variety of compounds to carriers for the production of antibodies.

The water soluble carbodiimide, 1-ethyl-3-(3-dimethylaminopropyl) carbodiimide (EDC) can be useful for conjugating an antiangiogenic peptide to a targeting peptide. Such conjugation requires the presence of an amino group, which can be provided, for example, by an antiangiogenic peptide, and a carboxyl group, which can be provided by the targeting peptide.

In addition to using carbodiimides for the direct formation of peptide bonds, EDC also can be used to prepare active esters such as N-hydroxysuccinimide (NHS) ester. The NHS ester, which binds only to amino groups, then can be used to induce the formation of an amide bond with the single amino group of the other second functional peptides. The use of EDC and NHS in combination is commonly used for conjugation in order to increase yield of conjugate formation (Bauminger and Wilchek, 1980).

Other methods for conjugating an antiangiogenic peptide to a targeting peptide also can be used. For example, sodium periodate oxidation followed by reductive alkylation of appropriate reactants can be used, as can glutaraldehyde crosslinking. However, it is recognized that, regardless of which method of producing a bifunctional peptide of this embodiment is selected, a determination must be made that the targeting molecule maintains its targeting ability and that the second functional peptide maintains its functional activity. Methods known in the art can confirm the activity of the bifunctional peptide.

Pharmaceutical compositions of the bifunctional peptide are prepared by conventional methods. The pharmaceutical composition or the bifunctional peptide is administered in accordance with procedures well known in the art.

In a sixth embodiment, the targeting peptide is conjugated to a small molecule, such as a therapeutic agent or a detectable agent. The detectable agent may be a radionuclide or an imaging agent (Wolfe et al., 2002), which allows detection or visualization. The type of detectable agent selected will depend upon the application. The therapeutic agent can be any biologically useful agent, such as a drug, such as a cytotoxic drug (e.g., as doxorubicin (Arap et al., 1998); see also U.S. Pat. No. 6,316,024), an antibiotic (such as ampicillin), an antiviral agent (such as ribavirin), an antisense nucleic acid molecule or a protease inhibitor that, when linked to targeting peptide of the invention, exerts its function at endothelial cells. The small molecules are conjugated to the targeting peptides as described above. For example, a drug such as doxorubicin is conjugated to a targeting peptide with 1-ethyl-3-(3-dimethyl-aminopropyl)carbodiimide hydrochloride (EDC) and N-hydroxysuccinimide (NHS) (Bauminger and Wilcheck, 1980; Arap et al., 1998). The conjugates are isolated and purified using conventional techniques. Pharmaceutical compositions of the targeting peptide-small molecule conjugate are prepared by conventional methods. The pharmaceutical composition or the conjugate is administered in accordance with procedures well known in the art.

In a seventh embodiment, the peptide is conjugated to liposome surfaces to target liposomes (Jaafari and Foldvari, 2002; Lestini et al., 2002), polylysine (Nah et al., 2002) or other polycation conjugates, and synthetic molecules. See also, for example, de Haan et al. (1996); Gorlach (1996); Benameur et al. (1995); Bonanomi et al. (1987); and Zekorn et al. (1995). In the description which follows, liposomes are utilized for exemplary purposes only. It is understood that this embodiment is not limited to liposomes.

Liposomes suitable for use in the composition of the present invention include those composed primarily of vesicle-forming lipids. Such a vesicle-forming lipid is one which (a) can form spontaneously into bilayer vesicles in water, as exemplified by the phospholipids, or (b) is stably incorporated into lipid bilayers, with its hydrophobic moiety in contact with the interior, hydrophobic region of the bilayer membrane, and its head group moiety oriented toward the exterior, polar surface of the membrane. Suitable liposomes are described in U.S. Pat. No. 6,316,024.

The liposomes may be prepared by a variety of techniques, such as those detailed in Szoka, F., Jr (1980), and specific examples of liposomes prepared in support of the present invention will be described below. Typically, the liposomes are multilamellar vesicles (MLVs), which can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

In one aspect, the pre-formed liposomes include a vesicle-forming lipid derivatized with a hydrophilic polymer to form a surface coating of hydrophilic polymer chains on the liposomes surface. Such a coating is preferably prepared by including between 1-20 mole percent of the derivatized lipid with the remaining liposome forming components, e.g., vesicle-forming lipids. Exemplary methods of preparing derivatized lipids and of forming polymer-coated liposomes have been described U.S. Pat. Nos. 5,013,556, 5,631,018 and 5,395,619, which are incorporated herein by reference. It will be appreciated that the hydrophilic polymer may be stably coupled to the lipid, or coupled through an unstable linkage which allows the coated liposomes to shed the coating of polymer chains as they circulate in the bloodstream or in response to a stimulus.

The therapeutic or diagnostic agent of choice can be incorporated into liposomes by standard methods, including (i) passive entrapment of a water-soluble compound by hydrating a lipid film with an aqueous solution of the agent, (ii) passive entrapment of a lipophilic compound by hydrating a lipid film containing the agent, and (iii) loading an ionizable drug against an inside/outside liposome pH gradient. Other methods, such as reverse evaporation phase liposome preparation, are also suitable. The therapeutic or diagnostic agents may be any agent conventionally included within liposomes, including nucleic acids. See, for example, U.S. Pat. No. 6,316,024.

The targeting conjugate is composed of (i) a lipid having a polar head group and a hydrophobic tail, e.g., a vesicle-forming lipid and any of those described above are suitable; (ii) a hydrophilic polymer attached to the head group of the vesicle-forming lipid; and (iii) a targeting peptide attached to the polymer. See, e.g., U.S. Pat. No. 6,316,024. The targeting peptide is covalently attached to the free distal end of the hydrophilic polymer chain, which is attached at its proximal end to a vesicle-forming lipid. There are a wide variety of techniques for attaching a selected hydrophilic polymer to a selected lipid and activating the free, unattached end of the polymer for reaction with a selected ligand, and in particular, the hydrophilic polymer polyethyleneglycol (PEG) has been widely studied (Allen et al., 1995; Zalipsky 1993; Zalipsky et al., 1994; Zalipsky et al., 1995; Zalipsky, 1995).

Generally, the PEG chains are functionalized to contain reactive groups suitable for coupling with, for example, sulfhydryls and amino groups present in the targeting peptides. Examples of such PEG-terminal reactive groups include maleimide (for reaction with sulfhydryl groups), N-hydroxysuccinimide (NHS) or NHS-carbonate ester (for reaction with primary amines), iodoacetyl (preferentially reactive with sulfhydryl groups) and dithiopyridine (thiol-reactive). Synthetic reaction schemes for activating PEG with such groups are set forth in U.S. Pat. Nos. 5,631,018, 5,527,528, and 5,395,619,

Pharmaceutical compositions of the targeted liposomes are prepared by conventional methods. The pharmaceutical composition or the targeted liposomes is administered in accordance with procedures well known in the art.

The peptides of the invention can be used to provide therapies for diseases, disorders or conditions associated with endothelial cells, including cancer and cardiovascular diseases such as diabetic retinopathy, macular degeneration, rheumatoid arthritis, psoriasis, plaque rupture, ischemic vascular diseases, wound healing, congestive heart failure, myocardial ischemia, reperfusion injury, peripheral arterial diseases, obesity and cardiovascular diseases such as ischemic heart disease, peripheral limb disease, vein graft stenosis and restenosis. That is, genes proteins, pharmaceuticals, radionuclides and other therapeutic or detecting agents can be directed to endothelial cells in those patients suffering from the particular disease, disorder or condition.

For example, chronic responses to endothelial cell injury include the development of intimal hyperplasia and arteriosclerosis, which limit the long-term success of coronary artery bypass grafting (Asimakopoulos and Taylor, 1998). The expression of integrins induced by the surgical trauma involved in coronary artery bypass grafting is associated with an inflammatory process characterized by the recruitment of neutrophils and monocytes (Takala et al., 1996). Migration of circulating neutrophils has been shown to be directed by endothelial cell expression of αᵥβ₃ integrin and this directed migration could be eliminated by neutralizing αᵥβ₃ integrin interactions with an RGD-containing peptide (Rainger et al., 1999). The increased expression of a, integrins has been described in isolated human saphenous vein segments (Meng et al., 1999) and rabbit vessels, and strategies aimed at inhibiting integrin interactions with a RGD-containing peptide have resulted in the reduction of neointima formation (Racanelli et al., 2000). Therefore, re-targeting of viral vector particles by the genetic incorporation of molecular ligands specifically recognized by upregulated vascular receptors during inflammation (Wickham et al., 1997) and vascular trauma is a strategy that might render significant advantages for adenoviral-mediated delivery of therapeutic transgenes. As shown herein, insertion of the targeting peptides of the present invention within the fiber HI loop resulted in enhanced gene transfer and expression in human umbilical vein endothelial cells.

The ex-vivo adenoviral transduction of veins before bypass grafting procedures offers the clear advantage of achieving maximal exposure of the entire vessel both intralumenally and to the outer adventitial layers. Additionally, the viral solution can be removed prior to transplantation thereby preventing undesired immunological responses caused by adenoviral particles released to the systemic circulation. Gene transfer of porcine jugular (Kibbe et al., 1999) and human saphenous veins transduced with adenoviral vectors carrying nitric oxide synthase (Cable et al., 1999) and tissue inhibitor of matrix metalloproteinase-1 (George et al., 1998) has established the feasibility of ex-vivo transduction and the clinical potential of adenoviral-mediated delivery of therapeutic transgenes. A recent randomized single-center clinical trial has demonstrated the potential of gene therapy to lower failure rates of human bypass vein grafting (Mann et al, 1999). Experimental strategies to maximize ex-vivo adenoviral vector delivery to veins such as the genetic engineering of the viral components responsible for cellular binding and internalization (fiber and/or penton base) should improve the efficiency of gene transfer and the therapeutic potential of these vectors.

It is understood that for each embodiment of the invention, one or more of the peptides may be used to enhance targeting to endothelial cells. Also, peptides of the invention may be used in combination with other targeting peptides that may or may not bind endothelial cells.

As described above, the targeting peptides of the invention can be linked to a moiety that is detectable external to the subject in order to perform an in vivo diagnostic imaging study or that is capable of delivering radioactivity to the tumor. Where the aim is to provide an image of the tumor, one will desire to use a diagnostic agent that is detectable upon imaging, such as a paramagnetic, radioactive or fluorogenic agent. Many diagnostic agents are known in the art to be useful for imaging purposes, as are methods for their attachment to peptides (see, e.g., U.S. Pat. Nos. 5,021,236 and 4,472,509, both incorporated herein by reference). In the case of paramagnetic ions, one might mention by way of example ions such as chromium (III), manganese (II), iron (III), iron (II), cobalt (II), nickel (I), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III). Moreover, in the case of radioactive isotopes for therapeutic and/or diagnostic application, one might mention ¹³¹iodine, ¹²³iodine, ^{99m}technicium, ¹¹¹indium, ¹⁸⁸rhenium, ¹⁸⁶rhenium, ⁶⁷gallium, ⁶⁷copper, ⁹⁰yttrium, ¹²⁵iodine, or ²¹¹astatine. Short-lived positron emission tomography (PET) isotopes, such as ¹⁸flourine, can also be used for labeling peptides for use in tumor diagnosis (Okarvi, 2001).

Where the aim is to treat the tumor, one will desire to use a radionuclide that will irradiate the tumor. Suitable radionuclides include ¹³¹iodine, ¹²³iodine, ^{99m}technicium, ¹¹¹indium, ¹⁸⁸rhenium, ¹⁸⁶rhenium, ⁶⁷gallium, ⁹⁰yttrium, ¹⁰⁵rhodium, ⁸⁹strontium, ¹⁵³samarium, ²¹¹astatine, ²¹²bismuth, ²¹³bismuth, ¹⁷⁷lutetium, ⁶⁷copper, ⁴⁷scandium, ¹⁰⁹palladium. Optimally, radionuclides are chosen for the specific application on the basis of physical and chemical properties such that (a) their decay mode and emitted energy are matched to the delivery site, (b) their half life and chemical properties are complementary to the biological processing and (c) production methods can yield the radionuclide at the necessary level of specific activity and radionuclide purity.

The incorporation of the radiometal into a peptide generally involves use of a chelate, specific to the particular metal, and a linker group to covalently attach the chelate to the targeting peptide, i.e., a the bifunctional chelate approach. The design of useful chelates is dependent on the coordination requirements of the specific radiometal. DTPA, DOTA, P₂S₂-COOH BFCA requirement for kinetic TETA, NOTA are common examples. The requirement for kinetic stability of the metal complex is often achieved through the use of multidentate chelate ligands with a functionalized arm for covalent bonding to some part of the peptide. Techniques for chelating radionuclides with proteins are well known in the art (see, e.g., WO 91/01144).

Pharmaceutical compositions containing a compound of the present invention as the active ingredient can be prepared according to conventional pharmaceutical compounding techniques. See, for example, Remington's Pharmaceutical Sciences, 18th Ed. (1990, Mack Publishing Co., Easton, Pa.). Typically, an antagonistic amount of active ingredient will be admixed with a pharmaceutically acceptable carrier. The carrier may take a wide variety of forms depending on the form of preparation desired for administration, e.g., intravenous, oral, parenteral or intrathecally. For examples of delivery methods see U.S. Pat. No. 5,844,077, incorporated herein by reference.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion.

The active agent is preferably administered in an therapeutically effective amount. By a “therapeutically effective amount” or simply “effective amount” of an active compound is meant a sufficient amount of the compound to treat the desired condition at a reasonable benefit/risk ratio applicable to any medical treatment. The actual amount administered, and the rate and time-course of administration, will depend on the nature and severity of the condition being treated. Suitable dosages can be readily determined by those of skill in the art. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use in humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the therapeutic agents of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

### EXAMPLES

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982; Sambrook et al., 1989; Sambrook and Russell, 2001; Ausubel et al., 1992; Glover, 1985; Anand, 1992; Guthrie and Fink, 1991; Harlow and Lane, 1988; Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

### Example 1

### Peptides Specific for Endothelial Cell Binding

TABLE 2
Peptide Sequences Specific for Endothelial Cells

| | |
|---|---|
| Sequence | SEQ ID NO: |

| | |
|---|---|
| CPDLHHHMC | 1 |
| CLGQHAFTC | 2 |
| CSSNTAPHC | 3 |
| CHVLPNGNC | 4 |
| CKPQYPSLC | 5 |
| CQTARTPAC | 6 |
| CNQSQPKHC | 7 |
| CTPSKISVC | 8 |
| CVSPGPRLC | 9 |
| CYALSGVPC | 10 |
| CKHPPQPFC | 11 |
| CHQSKPLLC | 12 |
| CPGPFSNWC | 13 |
| CPHKTHLPC | 14 |
| CVFPLSHYC | 15 |
| CNMIAPSSC | 16 |
| CTLGMQFQC | 17 |
| CTNPTGMLC | 18 |
| CSNMAPRSC | 19 |
| CSMAPNMSC | 20 |
| CSDLTMEAC | 21 |
| CPWPYKYSC | 22 |
| CFGGNFHRC | 23 |
| CLTTSQQTC | 24 |
| CTANSGSFC | 25 |
| CQEPLDESC | 26 |
| CQMSMFARC | 27 |
| CPLTPKAYC | 28 |
| CNNSHTALC | 29 |
| CLSSDITLC | 30 |
| CLTHGPKYC | 31 |
| CLGKDLRTC | 32 |
| CAPKTHPLC | 33 |
| CPTGLMKYC | 34 |
| CTWKAPLQC | 35 |
| CSHILGPSC | 36 |
| CLSTSQYSC | 37 |
| CXXPTPPXC | 44 |

### Example 2

### Construction of Adenoviral Vector Particles Specific for Endothelial Cells

The adenovirus Av3nBgPD1 is generated by genetically inserting the PD1 peptide (SEQ ID NO:1) into the HI loop of fiber knob in an adenovirus comprising the nuclear localized β-galactosidase reporter gene. In vitro transduction experiments are conducted to determine the binding characteristics of the PD1 targeted viral particle on primary human endothelial cells as well as three human carcinoma cell lines. The Av3nBgPD1 viral particle significantly enhances transduction on primary endothelial cells and H460 cells, a non-small cell carcinoma cell line. These data suggest that this peptide may have utility in targeting adenoviruses to vascular endothelial cells and some tumor cells.

Molecular retargeting of adenovirus particles is hypothesized to increase the number of viral ligand-receptor interactions on the cell membrane as well as the number of viral particles translocated to the cytoplasm of the targeted cells. The adenovirus fiber carboxy-terminus and the HI loop present in the fiber knob represent sites for the incorporation of short peptide motifs specifically recognized by cell surface receptors expressed by the target cells. It has been demonstrated that the HI loop of the fiber knob can be utilized for the insertion of short heterologous targeting peptides without disrupting fiber function (Krasnykh et al. 1998).

In this example, a nine amino acid peptide containing the amino acids CPDLHHHMC (SEQ ID NO:1) is genetically incorporated into the fiber HI loop. This peptide (SEQ ID NO:1) is referred to as PD1. An adenoviral vector particle called Av3nBgPD1 is generated, which comprises the reporter gene β-galactosidase, contains the PD1 peptide within the fiber knob and has the genes in the E1 and E2a region deleted (Gorziglia et al., 1996). The targeted adenoviral vector particle is then analyzed for its ability to enhance transduction to primary endothelial cells as well as several human carcinoma cell lines.

Cell Culture

S8 cells are A549 cells stably transfected with adenoviral E1 and E2a genes under separate dexamethasone-inducible promoters (Gorziglia et al., 1996). S8 cells are cultured in IMEM (Biofluids, Rockville, Md.) with 10% heat inactivated fetal bovine serum (HIFBS). For virus production, the cells are cultured with 0.3 uM dexamethasone to induce the expression of E1 and E2a genes. H460 cells are a human non-small cell lung carcinoma (ATCC, Manassas, Va.) and are cultured in RPMI Medium 1640 (Life Technologies, Gaithersburg, Md.) with 10% HIFBS. PC3 cells are a human prostate carcinoma cell line (ATCC) and are cultured in RPMI Medium 1640 with 10% HIFBS. HeLa cells are a human cervical carcinoma cell line (ATCC) and are cultured in DMEM with 10% HIFBS. Primary human endothelial cells, in particular Human Umbilical Vein Endothelial Cells (HUVECs) are obtained from the Clonetics Corporation (Walkerville, Md.: AC-7018). The cells are cultured in the recommended medium.

Two-plasmid System Used to Generate Recombinant Adenovirus

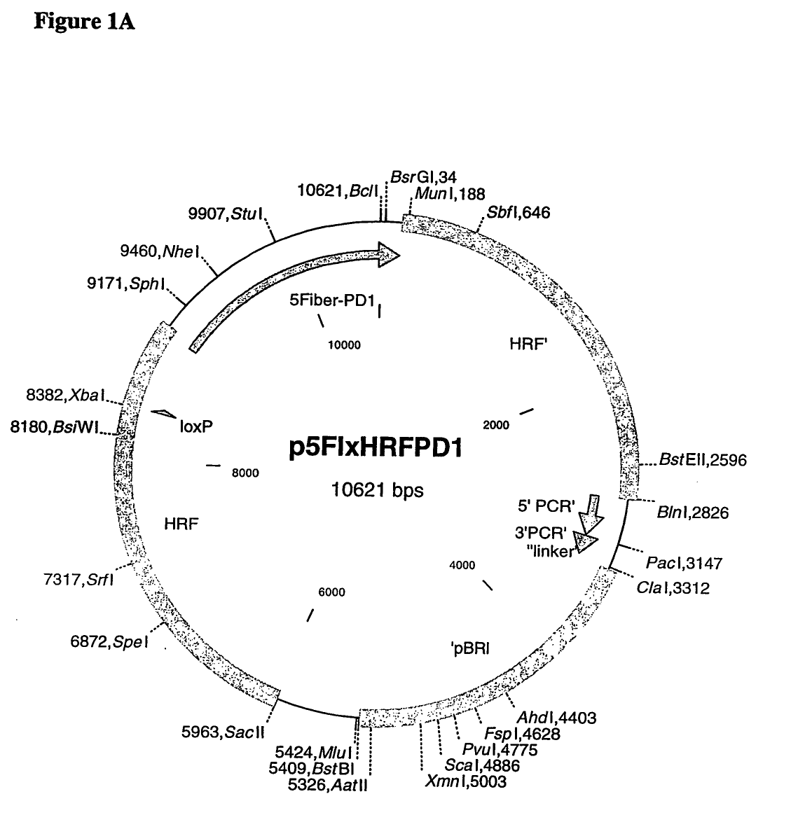

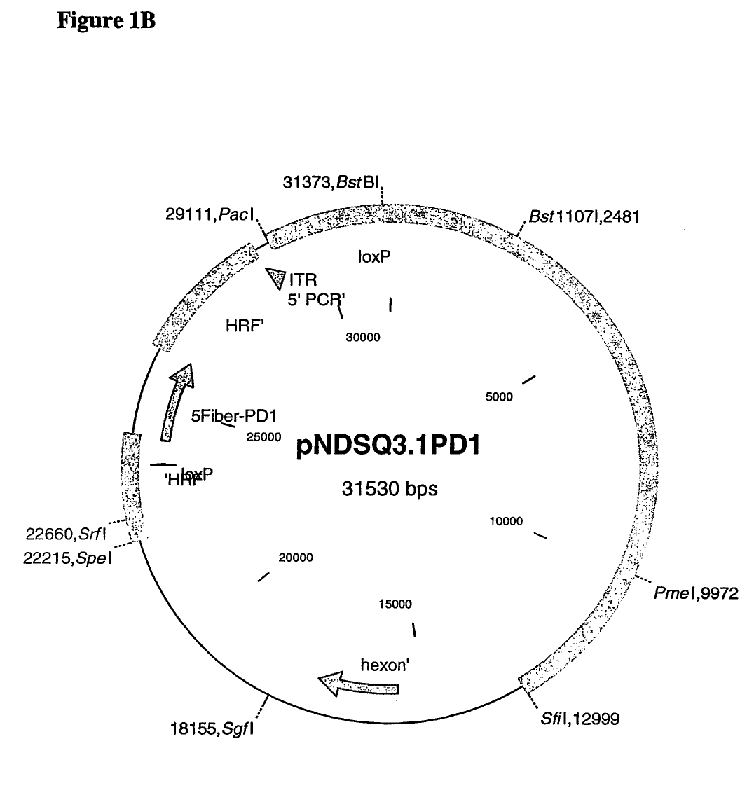

Av3nBgPD1, an adenovirus encoding nuclear localized β-galactosidase with the PD1 peptide in the HI loop of the fiber knob is generated by a rapid two plasmid system. Briefly, pNDSQ3.1PD1, a plasmid containing the 29 Kb right hand portion of the adenovirus serotype 5 genome which contains the modified fiber gene with the PD1 peptide inserted in the HI loop is linearized with Cla1. A second plasmid, pAdmireRSVnBg, encodes the left end of the adenoviral genome containing the RSV promoted nuclear localized β-galactosidase cDNA and overlapping sequences to allow for homologous recombination. The pAdmire plasmid is digested with PacI and Sal I to release the ITR and E. coli sequences in the plasmid. The digested plasmids are cotransfected into induced S8 cells (Gorziglia et al., 1996) using the cationic lipid Lipofectamine plus system (Life Technologies (LTI), Gaithersburg, Md.). Transfected S8 cells will support the propagation of the resulting recombinant adenovirus. FIG. 1B shows the pNDSQ3.1PD1 plasmid for generating Av3nBgPD1 adenovirus.

Construction of Av3nBgPD1

The PD1 sequence, CPDLHHHMC (Seq ID NO:1), is inserted into the HI loop of Ad5 fiber, between D544 and T545 in a E1/E2a deleted adenoviral vector encoding the nuclear localized β-galactosidase gene. The insertion is accomplished by annealing oligonucleotides containing the PD1 peptide and overhangs for Bcl1 and BsrG1 sites that are engineered into the HI loop of fiber to enable ligand insertion. The oligonucleotide sequences are shown below.

34 mer, 5.0 μg/μl

(SEQ ID NO:38)

5′-GATCAATGTCCTGACCTACACCACCACATGTGTT-3′
34 mer, 4.0 μg/μl

(SEQ ID NO:39)

5′-GTACAACACATGTGGTGGTGTAGGTCAGGACATT-3′

Each of the oligonucleotides are phosphorylated in separate reactions by combining 5 μl oligo, 10 μl 5× Forward Buffer (LTI), 0.5 μl 100 mM ATP, 2 μl kinase (LTI) and 32.5 μl H₂O for a total volume of 50 μl. The reactions are incubated at 37° C. for one hour. The phosphorylated oligos are then annealed by combining both 50 μl kinase reactions, 96 μl TE pH 7.4 and 4 μl 5M NaCl for a total volume of 200 μl. The anneal reaction is boiled for three minutes, then allowed to slowly cool to room temperature. The DNA is then precipitated and ligated into the BclI and BsrGI sites of p5FloxHRPRGD to generate p5FloxHRFPD1 (FIG. 1A). This plasmid contains the PD1 coding sequences inserted into the coding sequence of the HI loop of fiber and is flanked by the unique restriction sites BclI and BsrGI to allow cloning of other peptide ligand coding sequences into this location of the fiber gene. In addition to the Ad5 fiber gene, this plasmid also contains approximately 8000 bp from the right end of the Ad5dl327 viral genome. The final pNDSQ3.1subP plasmid is generated by ligating the isolated SpeI/PacI fragment from p5FloxHRFPD1 into pNDSQ3.1 creating the plasmid pNDSQ3.1PD1. The correct pNDSQ3.1PD1 plasmid (FIG. 1B) is confirmed by restriction analysis and sequence analysis.

A six well tissue culture plate is seeded with 5×10⁵ S8 cells (Gorziglia et al., 1996) per well grown in IMEM containing 10% HIFBS and 0.33 μM dexamethasone approximately 24 hours prior to transfection. The pNDSQ3.1PD1 plasmid is digested with ClaI, extracted with phenol:chloroform:isoamylalcohol (25:24:1), and then DNA is precipitated with ethanol and 3M sodium acetate. The DNA is pelleted and resuspended in dH₂O to a concentration of 1 μg/μl. The pAdmireRSVnBg plasmid is processed the same way, except the DNA is digested with the PacI and SalI restriction endonucleases. The digested pAdmireRSVnBg plasmid is resuspended in dH₂O to a concentration of 0.5 μg/μl. The Lipofectamine plus cationic lipid system (Life Technologies, Gaithersburg, Md.) is used to co-transfect the plasmids into dexamethasone induced S8 cells as follows. For each duplicate reaction, 1 μg of ClaI digested pNDSQ3.1PD1 and 0.5 μg of Pac I/Sal I digested pAdmireRSVnBg is added to a mixture of 6 μl plus reagent and 92 μl opti-MEM 1 media. The 100 μl plus/DNA solution is incubated at room temperature for 15 minutes. In a separate tube, 4 μl lipofectamine and 100 μl opti-MEM1 media are combined. After incubation the DNA mixture is added to the lipofectamine solution, mixed, and allowed to incubate at room temperature for an additional 15 minutes. The S8 cell monolayer is washed with opti-MEM1 media (Life Technologies, Gaithersburg, Md.) and aspirated. The DNA transfection is added to each well with 800 μl of opti-MEM 1 media and the 200 μl transfection complex. The reagents are then incubated at 37° C. in the CO₂ incubator for 3 to 5 hours. The transfection mix is aspirated and 2 ml of growth media supplemented with 0.33 μM dexamethasone is added to each well. The plate is incubated for 7 days at 37° C. 5% CO₂.

The transfected S8 cells are monitored for the appearance of cytopathic effect (CPE) which is a rounding of the cells into grape-like clusters as a result of virus production. Amplification is conducted as follows: the cells are detached from the well using a cell lifter, and the cells plus media are transferred into a 15 ml conical tube. To disrupt the cells, three rounds of freeze-thaw cycles are conducted with vigorous vortexing after each thaw. The cellular debris is pelleted, and 600 μl of the crude viral lysate (CVL) is applied per well of a monolayer of 5×10⁵ induced S8 cells seeded in a 6 well tissue culture plate. The CVL is rocked in a 37° C. incubator for 3 hours. 2 ml of growth media plus dexamethasone is added to each well and the plate is placed in the 37° C. CO₂ incubator. A second round of amplification is conducted after 7 days. When CPE is observed the virus is scaled up on fifteen 150 cm plates of induced S8 cells. A virus prep of Av3nBgPD1 is generated by standard CsCl centrifugation. The virus particle number per ml is determined spectrophotometrically as described (Mittereder et. al. 1996).

In Vitro Transduction Analysis

The transduction efficiency of Av3nBgPD1 is surveyed using primary human umbilical vein endothelial cells (HUVEC) and on three human carcinoma cell lines including HeLa, PC3, and H460 cells. Each cell type is transduced with the chimeric fiber containing virus, Av3nBgPD1 or the wildtype fiber control virus, Av3nBg. HUVECs were transduced with 0, 10, 100, and 1000 total particles per cell (PPC). The three carcinoma cell lines are transduced with 0, 50, 100, and 1000 total particles per cell (PPC). All cell lines are transduced for 1 hour at 37° C. in a total volume of 0.2ml of culture medium containing 2% HI-FBS, then 1 ml of complete medium containing 10% HIFBS is added. The cells are incubated for an additional 24 hours to allow for the adenoviral-mediated β-galactosidase gene expression. The cell monolayers are then fixed with 0.5% glutaraldehyde in PBS followed by incubation with X-gal stain for approximately 24 hours. The X-gal stain consists of 1 mg/ml 5-bromo-4-chloro-3-indolyl-β-D-galactosidase (X-gal, 50 mg/ml stock made up in DMSO), 5mM Potassium Ferrocyanide, 5 mM Potassium Ferricyanide and 2mM MgCl₂ in PBS. The stain is removed and the cell monolayers are washed with PBS. The percentage of transduction is determined by light microscopy by counting the number of positively transduced blue cells per field as described previously (Stevenson, et. al, 1997).

Results and Discussion

Synthetic oligonucleotides encoding PD1 peptide:CPD MC (SEQ ID NO:1) are designed to genetically insert this peptide into the fiber knob HI loop between amino acids 544 and 545. Co-transfections are carried out using pNDSQ3.1PD1 and padmireRSVnBg to generate Av3nBgPD1, which contains the nuclear-targeted β-galactosidase cDNA and the PD1 peptide in the fiber knob.

The transduction efficiency of Av3nBgPD1 is surveyed on primary human endothelial cells and three separate human carcinoma cell lines. Cells are transduced with the PD1 chimeric fiber containing Av3nBhPD1 virus or the control virus, Av3nBg. The results of exemplary experiments performed according to the above procedures are shown in Table 3 below.

TABLE 3
Enhancement of Adenoviral Transduction Using the PD1 Peptide

| Cell Line | ^{#}PPC | Av3nBgPD1 | Av3nBg Control |
|---|---|---|---|
| HUVEC | 10 | 1.83 ± 0.78 | 1.22 ± 0.86 |
| | 100 | 6.44 ± 1.45 | 4.93 ± 1.88 |
| | 1000 | 42.1 ± 6.5* | 21.7 ± 4.0 |
| HeLa | 50 | 21.1 ± 2.9 | 25.7 ± 5.5 |
| | 100 | 44.9 ± 8.4 | 52.3 ± 10.4 |
| | 1000 | 79.2 ± 5.2 | 85.3 ± 3.3 |
| PC3 | 50 | 1.18 ± 1.02 | 0.59 ± 0.83 |
| | 100 | 2.26 ± 1.2 | 2.07 ± 0.73 |
| | 1000 | 8.2 ± 2.6 | 6.8 ± 2.1 |
| H460 | 50 | 5.71 ± 1.41* | 2.28 ± 0.79 |
| | 100 | 14.7 ± 2.76* | 7.93 ± 0.93 |
| | 1000 | 66.9 ± 7.8* | 39.8 ± 9.4 |

Each cell type was transduced with the indicated dose of each vector particle. ^{#}PPC, particles per cell. The data represent the mean percentage±standard deviation from triplicate determinations from a representative experiment. *, Significantly different from Av3nBg control according to an unpaired, two-tailed t-test (P<0.0001).The percent transduction as a function of adenovirus dose is shown for primary HUVECs. Both vector particles transduced human EC in a dose dependent manner. However, at the dose of 1000 PPC there was a statistically significant increase using Av3nBgPD1 with 42.1% positive cells achieved compared to 21.7% obtained with Av3nBg. PD1-mediated transduction was assessed on HeLa, PC3 and H460 carcinoma cell. Both HeLa and PC3 cells were equally susceptible to transduction with both vector particles indicating that PD1 offered no advantage. In comparison, Av3nBgPD1 significantly enhanced adenoviral gene transfer to H460 cells at the 1000 PPC dose.

As shown in this example, PD 1 (SEQ ID NO:1), enhances transduction of endothelial cells. This example illustrates the incorporation of the PD1 peptide (SEQ ID NO:1) into an adenoviral vector particle and results in an increased percent transduction of endothelial cells. It is understood that while this example illustrates one embodiment of the invention with the peptide of SEQ ID NO:1, any of the targeting peptides of the invention (SEQ ID NOs:2-37 & 44; Table 2) may be used in a similar manner.

### Example 3

### Preparation of sCAR Conjugated to Targeting Peptide

To construct expression plasmids encoding a targeting peptide at the carboxy-terminus of sCAR, pairs of complementary oligonucleotides are synthesized and annealed to form a DNA duplex encoding the desired targeting peptide. The DNA duplexes are designed to contain NotI compatible overhangs on both ends so the fragment can be inserted into the NotI site of pCI-neo-sCARb (WO 02/29072). The peptide CPDLHHHMC (SEQ ID NO:1) is fused to the end of sCAR or incorporated at a location which allows for specific binding of the targeting peptide to the target cell. The oligonucleotides that are synthesized to generate CPDLHHHMC (SEQ ID NO:1) are as follows:

(SEQ ID NO:40)

5′-GGCCTGTCCTGATCTTCATCATCATATGTGTGC-3′
and

(SEQ ID NO:41)

5′-GGCCGCACACATATGATGATGAAGATCAGGACA-3′.

A plasmid encoding trimerized sCAR and a plasmid encoding a trimerized version of sCAR containing the CPDLHHHMC (SEQ ID NO:1) targeting peptide are constructed as described in WO 02/29072. The sCAR conjugated to CPDLHHHMC (SEQ ID NO:1) is prepared and purified as described in WO 02/29072. A complex of an adenoviral vector particle and the sCAR conjugated targeting peptide is prepared as described in WO 02/29072. The complex binds selectively to endothelial cells. It is understood that this example illustrates one embodiment of the invention with the peptide of SEQ ID NO:1, but that any of the targeting peptides of the invention (SEQ ID NOs:2-37 & 44) may be used in a similar manner.

### Example 4

### Preparation of Retroviral Particle with Targeting Modified Surface Protein

A retroviral particle having a modified surface protein, in which the modification is the incorporation of the targeting peptide, CPDLHHHMC (SEQ ID NO:1), is prepared as described in WO 98/44938. The nucleotide sequence encoding this target peptide TGTCCTGATCTTCATCATCATATGTGT (SEQ ID NO:42) is used in making the nucleic acid encoding the modified surface protein. The retroviral particle binds selectively to endothelial cells. It is understood that while this example illustrates one embodiment of the invention with the peptide of SEQ ID NO:1, any of the targeting peptides of the invention (SEQ ID NOs:2-37 & 44) may be used in a similar manner.

### Example 5

### Preparation of Growth Factor-Targeting Peptide Fusion Protein

A nucleic acid encoding a fusion protein of vascular endothelial growth factor and CPDLHHHMC (SEQ ID NO:1) and an expression vector containing this nucleic acid are prepared as described in WO 00/06195. The fusion protein is expressed in host cells transfected with the expression vector and is isolated using conventional techniques. The fusion protein binds selectively to endothelial cells. It is understood that while this example illustrates one embodiment of the invention with the peptide of SEQ ID NO:1, any of the targeting peptides of the invention (SEQ ID NOs:2-37 & 44) may be used in a similar manner.

### Example 6

### Preparation of Bifunctional Peptide

A bifunctional peptide is prepared containing CPDLHHHMC (SEQ ID NO:1) as the targeting domain and _{D}(KLAKLAKKLAKLAK) (SEQ ID NO:43) as the pro-apoptotic domain, in which all of the amino acid residues in the pro-apoptotic domain are the D-enantiomers. The synthesis of the bifunctional peptide with a glycine-glycine bridge between the two domains is performed using conventional solid phase techniques. The bifunctional peptide retains binding selectivity to endothelial cells. It is understood that while this example illustrates one embodiment of the invention with the peptide of SEQ ID NO:1, any of the targeting peptides of the invention (SEQ ID NOs:2-37 & 44) may be used in a similar manner.

### Example 7

### Conjugation of Targeting Peptide and a Small Molecule

Doxorubicin hydrochloride (1 molar equivalent) is suspended in dimethylformamide (DMF) containing diisopropylamine (2 molar equivalents). N-hydroxysuccinimidyl-maleimidopropionate (1 molar equivalent) is added and incubated for 20 min. The thiol-containing the targeting peptide CPDLHHHMC (SEQ ID NO:1) (either as a cysteine or as amino-terminal 3-mercaptopropionic acid solubilized in DMF) is then added to this reaction mixture, followed by a 20-min incubation. The acceptance criteria for the peptide and conjugates is HPLC purity of >98% in accordance with the molecular weight and fragmentation pattern for mass spectrometry.

Alternatively, doxorubicin hydrochloride is suspended in DMF containing diisopropylamine. Succinic anhydride (1 molar equivalent) dissolved in DMF is added and incubated for 20 min. The resulting doxorubicin hemisuccinate is then activated by addition of benzotriazol-1-yl-oxopyrrolidinephosphonium hexafluorophosphate (1.1 molar equivalents) dissolved in DMF. The targeting peptide CPDLHHHMC (SEQ ID NO:1) is then added to the reaction mixture after 5 min of activation and left for another 20 min for coupling. Further processing and purity check of the conjugate is performed as described above. The small molecule doxorubicin attached to the targeting peptide selectively binds to endothelial cells. It is understood that while this example illustrates one embodiment of the invention with the peptide of SEQ ID NO:1, any of the targeting peptides of the invention (SEQ ID NO:2-37 & 44) may be used in a similar manner.

### Example 8

### Preparation of Targeted Liposome

Liposomes are prepared by mixing partially hydrogenated soy-bean phosphatidylcholine (PHPC, iodine value of 35, Lipoid (Ludwigshafen, Germany)), cholesterol (Croda (Fullerton, Calif.)) and mPEG-DSPE (prepared as described in Zalipsky, 1993) at a molar ratio of 55:40:3 in chloroform and/or methanol in a round bottom flask. The solvents are removed by rotary evaporation, and the dried lipid film produced is hydrated with either sodium phosphate buffer (10 mM, 140 mM NaCl, pH 7) or HEPES buffer (25 mM, 150 mM NaCl, pH 7) to produce large multilamellar vesicles. The resulting vesicles are passed repeatedly under pressure through 0.2, 0.1 and 0.05 m pore size polycarbonate membranes, until the average size distribution for the diameter (monitored by dynamic light scattering using a Coulter N4MD (Hialeah, Fla.)) is approximately 100 nm (U.S. Pat. No. 6,316,024 B1).

Targeting conjugates of CPDLHHHMC (SEQ ID NO:1)-PEG-DSPE (DSPE: distearoyl phosphatidylethanolamine) are prepared according to Zalipsky et al. (1997).

The pre-formed liposomes are incubated at either 25° C. or 37° C. with 1.2 mole percent of the targeting conjugate. At various time points, targeting conjugates (micelles) are separated from inserted targeting conjugates (liposomes) by size exclusion chromatography. For the sialyl-Lewis^{x}-PEG-DSPE conjugate, a Biogel ASOM column equilibrated with 10 mM sodium phosphate, 140 mM sodium chloride, and 0.02% NaN.sub.3 at pH 6.5 is used. For CPDLHHHMC (SEQ ID NO:1)-PEG-DSPE conjugate, a Sepharose 4B column is used with 10% sucrose and 10 mM HEPES at pH 7.0 as eluent.

The collected fractions (1 mL) from the size exclusion chromatograph are diluted 1:10 in methanol, and analyzed for ligand content by HPLC (Shimadzu and Rainin systems). Incorporating the targeting peptide into the liposome causes the liposome to selectively bind endothelial cells. It is understood that while this example illustrates one embodiment of the invention with the peptide of SEQ ID NO:1, any of the targeting peptides of the invention (SEQ ID NO:2-37 & 44) may be used in a similar manner.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

Anand, R. (1992). Techniques for the Analysis of Complex Genomes, Academic Press, New York.
Angelini, G. D. and Newby, A. C. (1989). The future of saphenous vein as a coronary artery bypass conduit. Eur Heart J 10:273-280.
Allen et al. (1979). Acta Crystallogr Section B 35:2331.
Allen, T. M. et al. (1995). A new strategy for attachment of antibodies to sterically stabilized liposomes resulting in efficient targeting to cancer cells. Biochemicia Biophysica Acta 1237:99-108.
Arap, W. et al. (1998). Cancer treatment by targeted drug delivery to tumor vasculature in mouse model. Science 279:377-380.
Arap, W. et al. (2002). Targeting the prostate for destruction through a vascular address. Proc Natl Acad Sci USA 99:1527-1531.
Asimakopoulos, G. and Taylor, K. M. (1998). Effects of cardiopulmonary bypass on leukocyte and endothelial adhesion molecules. Ann Thorac Surg 66:2135-2144.
Ausubel, F. M. et al. (1992). Current Protocols in Molecular Biology, John Wiley & Sons, New York.
Bai, M. et al. (1993). Mutations that alter an Arg-Gly-Asp (RGD) sequence in the adenovirus type 2 penton base protein abolish its cell-rounding activity and delay virus reproduction in flat cells. J Virol 67:5198-5205.
Bauminger, S. and Wilchek, M. (1980). The use of carbodiimides in the preparation of immunizing conjugates. Meth Enzymol 70:151-159.
Benameur, H. et al. (1995). Liposome-incorporated dexamethasone palmitate inhibits in-vitro lymphocyte response to mitogen. J Phar Pharmacol 47:812-817.
Bergelson, J. M. et al. (1997). Isolation of a common receptor for Coxsackie B viruses and adenoviruses 2 and 5. Science 275:1320-1323.
Berkowitz et al. (2001). J. Virol. 7:3371-3382.
Blondelle et al. (1995). Trends Anal Chem 14:83-92.
Bonanomi, M. H. et al. (1987). Fate of different kinds of liposomes containing dexamethasone palmitate after intra-articular injection into rabbit joints. J Microencapsul 4:189-200.
Cable, D. G. et al. (1999). The role of gene therapy for intimal hyperplasia of bypass grafts. Circulation 100:11392-11396.
Cameron, A. et al. (1996). Coronary bypass surgery with internal-thoracic-artery grafts—effects on survival over a 15-year period [see comments]. N Engl J Med 334:216-219.
Chartier, C. et al., (1996). Efficient generation of recombinant adenovirus vectors by homologous recombination in Escherichia coli. J Virol 70:4805-4810.
Curiel, D. T. (1991). Adenovirus enhancement of transferrin-polylysine-mediated gene delivery. Proc Natl Acad Sci USA 88:8850-8854.
Curiel, D. T. et al. (1992). High-efficiency gene transfer mediated by adenovirus coupled to DNA-polylysine complexes. Human Gene Therapy 3:147-154.
Cumis, F. et al., (2002). Differential binding of drugs containing the NGR motif to CD13 isoformns in tumor vesels, epithilia, and myeloid cells. Cancer Research 62:867-874.
Curran, T. et al. (1982). FBJ murine osteosarcoma virus: identification and molecular cloning of biologically active proviral DNA. J Virol 44:674-682.
Dai, Y. (1995). Cellular and humoral immune responses to adenoviral vectors containing factor IX gene: tolerization of factor IX and vector antigens allows for long-term expression. Proc Natl Acad Sci USA 92:1401-1405.
de Haan, A. et al. (1996). Mucosal immunoadjuvant activity of liposomes: role of alveolar macrophages. Immunology 89:488-493.
DiMaio, D. et al. (1982). Bovine papillomavirus vector that propagates as a plasmid in both mouse and bacterial cells. Proc Natl Acad Sci USA 79:4030-4034.
Douglas et al. (2001). Hum Gene Ther. 12:401-413.
Ecker, D. J. and Crooke, S. T. (1995). Design, synthesis and in vitro testing of methotrexate carrier conjugates linked via oligopeptide spacers. Biotechnology 13:351-360.
Ellerby, H. M. et al. (1999). Anti-cancer activity of targeted pro-apoptotic peptides. Nature Medicine 9:1032-1038.
Ferkol, T. et al. (2000). Transport of bifunctional proteins across respiratory epithelial cells via the polymeric immunoglobulin receptor. Am J Respir Crit Care Med 161:944-51.
Fitzpatrick and Garnett (1995). Design, synthesis and in vitro testing of methotrexate carrier conjugates linked via oligopeptide spacers. Anticancer Drug Des 10: 1-9.
Freshney, R. I. (1987). Culture of Animal Cells, Alan R. Liss, Inc.
Garvey et al. (1990). Virology 175:391-409.
Gazit, A. et al. (1986). Mammalian cell transformation by a murine retrovirus vector containing the avian erythroblastosis virus erbB gene. J Virol 60:19-28.
Geller, A. J. et al. (1988). A defective HSV-1 vector expresses Escherichia coli beta-galactosidase in cultured peripheral neurons. Science 241:1667-1669.
George, S. J. et al. (1998). Adenovirus-mediated gene transfer of the human TIMP-1 gene inhibits smooth muscle cell migration and neointimal formation in human saphenous vein. Hum Gene Ther 9:867-877.
Glover, D. (1985). DNA Cloning, I and II, Oxford Press, London.
Gollan, T. J. and Green, M. R. (2002). Selective targeting and inducible destruction of human cancer cells by retroviruses with envelope proteins bearing short peptide ligands. J Virol 76:3564-3569.
Goodman and Ro (1995). Peptidomimetics for Drug Design, in Burger's Medicinal Chemistry and Drug Discovery, 5^{th} Ed., Vol. 1, Wolff, M. E. (ed), pp. 803-861, John Wiley & Sons, New York.
Gordon, E. M. et al. (1994). Applications of combinatorial technologies to drug discovery. 2. Combinatorial organic synthesis, library screening strategies, and future directions. J Med Chem 37:1385-1401.
Gorlach, A. (1996). [Experiences in the changes of biotechnology in veterinary practice] Dtsch Tierarztl Wochenschr 103:312-315.
Gorziglia, M. I. et al. (1996). Elimination of both E1 and E2 from adenovirus vectors further improves prospects for in vivo human gene therapy. J Virol 70:4173-4178.
Grifman, M. et al., (2001). Incorporation of tumor-targeting peptides into recombinant adeno-associated virus capsids. Molecular Therapy 3:964-975.
Guthrie, G. and Fink, G. R. (1991). Guide to Yeast Genetics and Molecular Biology, Academic Press, New York.
Hames, B. D. and Higgins, S. J. (eds.) 1984. Nucleic Acid Hybridization.
Hames, B. D. and Higgins, S. J. (eds.) 1984. Transcription and Translation.
Harbury, P. B. et al. (1993). A switch between two-, three-, and four-stranded coiled coils in GCN4 leucine zipper mutants. Science 262:1401-1407.
Harlow, E. and Lane, D. (1988). Antibodies: A Laboratory Manual, Cold Spring Harbor Laboratory, Cold Spring Harbor, N.Y.
Hay, C. M. et al. (2001). Enhanced gene transfer to rabbit jugular veins by an adenovirus containing a cyclic RGD motif in the HI loop of the fiber knob. J Vasc Res 38:315-323.
Henry, L. J. et al. (1994). Characterization of the knob domain of the adenovirus type 5 fiber protein expressed in Escherichia coli. J. Virol. 68(8):5239-5246.
Hermanson (1996). Bioconjugate Techniques, Academic Press, New York.
Hogan et al. (1986). Manipulating the Mouse Embryo, Cold Spring Harbor Laboratory Press, Cold Spring Harbor, N.Y.
Hong, S. S. et al. (1993). Adenovirus type 5 fiber knob binds to MHC class I alpha2 domain at the surface of human epithelial and B lymphoblastoid cells. EMBO J 16:2294-2306.
Horton, R. M. et al. (1990). Gene splicing by overlap extension: tailor-made genes using the polymerase chain reaction. Biotechniques 8:528-535.
Horwitz, M. S. (1996). “Adenoviruses” In Fields Virology, Third Edition, Fields, B. N. et al. (eds), Lippincott-Raven Publishers: Philadelphia, Pa.
Huard, J. et al. (1995). Herpes simplex virus type I vector mediated gene transfer to muscle. Gene Therapy 2:385-392.
Immobilized Cells and Enzymes (1986), IRL Press.
Jaafari, M. R. and Foldvari, M. (2002). Targeting of liposomes to melanoma cells with high levels of ICAM-1 expression through adhesive peptides from immunoglobulin domains. J Pharm Sci 91:396-404.
Jakoby, W. B. and Pastan, I. H. (eds.) (1979). Cell Culture. Methods in Enzwnology Vol. 58, Academic Press, Inc., New York.
Kalan, J. M. and Roberts, W. C. (1990). Morphologic findings in saphenous veins used as coronary arterial bypass conduits for longer than 1 year: necropsy analysis of 53 patients, 123 saphenous veins, and 1865 five-millimeter segments of veins. Am Heart J 119:1164-1184.
Kass-Eisler, A. et al. (1993). Quantitative determination of adenovirus-mediated gene delivery to rat cardiac myocytes in vitro and in vivo. Proc Natl Acad Sci USA 90:11498-11502.
Kavanaugh, M. P. et al. (1994). Cell-surface receptors for gibbon ape leukemia virus and amphotropic murine retrovirus are inducible sodium-dependent phosphate symporters. Proc Natl Acad Sci USA 91:7071-7075.
Kibbe, M. R. et al. (1999). Optimization of ex vivo inducible nitric oxide synthase gene transfer to vein grafts. Surgery 126:323-329.
Klein et al. (1985). Biochim. Biophys Acta 815:468.
Krasnykh et al. (1998). Characterization of an adenovirus vector containing a heterologous peptide epitope in the HI loop of the fiber knob. J. Virol. 72(3):1844-1852.
Le Gal La Salle, G et al. (1993). An adenovirus vector for gene transfer into neurons and glia in the brain. Science 259:988-990.
Lestini, B. J. et al., 2002. Surface modification of liposomes for selective cell targeting in cardiovascular drug delivery. J Control Release 78:235-247.
Loop, F. D. et al. (1986). Influence of the internal-mammary-artery graft on 10-year survival and other cardiac events. N Engl J Med 314:1-6.
Maniatis, T. et al. (1982). Molecular Cloning: A Laboratory Manual, Cold Spring Harbor Laboratory, Cold Spring Harbor, N.Y.
Mann, M. J. et al. (1999). Ex-vivo gene therapy of human vascular bypass grafts with E2P decoy: the PREVENT single-centre, randomised, controlled trial. Lancet 354:1493-1498.
Mayer and Walker (eds.) (1987). Immunochemical Methods in Cell and Molecular Biology, Academic Press, London.
McKay, I. and Leigh, I. (eds) (1993). Growth Factors: A Practical Approach, IRL Press, Oxford.
Meng,. X. et al. (1999). Mechanical stretching of human saphenous vein grafts induces expression and activation of matrix-degrading enzymes associated with vascular tissue injury and repair. Exp Mol Pathol 66:227-237.
Methods In Enzymology, Academic Press, Inc., N.Y.
Miller, J. H. and Calos, M. P. (eds.) (1987). Gene Transfer Vectors for Mammalian Cells, Cold Spring Harbor Laboratory.
Miller, A. D. (1992). Retroviral vectors. Curr Top Microbiol Immunol 158:1-24.
Mittereder, N. et al. (1994). Evaluation of the efficacy and safety of in vitro, adenovirus-mediated transfer of the human cystic fibrosis transmembrane conductance regulator cDNA. Hum. Gene Ther 5:717-729.
Miyoshi et al. (1999). Virol. 72:8150-8157.
Morin, J. E. et al. (1987). Recombinant adenovirus induces antibody response to hepatitis B virus surface antigen in hamsters. Proc Natl Acad Sci USA 84:4626-4630.
Muzyczka, N. (1994). Adeno-associated virus (AAV) vectors: will they work? J Clin Invest 94:1351.
Nah, J. W. et al. (2002). Artery wall binding peptide-poly(ethylene glycol)-grafted-poly(L-lysine)-based gene delivery to artery cells. J Control Release 78:273-284.
Nemerow, G. R. and Stewart, P. L. (1999). Role of alpha(v) integrins in adenovirus cell entry and gene delivery. Microbiol Mol Biol Rev 63:725-734.
Newby, A. C. and Baker, A. H. (1999). Targets for gene therapy of vein grafts. Curr Opin Cardiol 14:489-494.
Okayama, H. et al. (1985). Bacteriophage lambda vector for transducing a cDNA clone library into mammalian cells. Mol Cell Biol 5:1136-1142.
Olive, B. et al. (1997). An automated classification of the structure of protein loops. Journal of Molecular Biology 266:814-830.
Perbal, B. (1984). A Practical Guide to Molecular Cloning.
Quantin, B. et al. (1992). Adenovirus as an expression vector in muscle cells in vivo. Proc Natl Acad Sci USA 89:2581-2584.
Racanelli, A. L. et al. (2000). Inhibition of neointima formation by a nonpeptide alpha(v)beta(3) integrin receptor antagonist in a rabbit cuff model. J Cell Biochem 77:213-220.
Rainger, G. E. et al. (1999). Neutrophils sense flow-generated stress and direct their migration through alphaVbeta3-integrin. Am J Physiol 276:H858-H864.
Reminigton's Pharmaceutical Sciences, 18th Ed., Mack Publishing Co., Easton, Pa., 1990.
Riott (1988). Essential Immunology, 6th Ed., Blackwell Scientific Publications, Oxford.
Rosenfeld, M. A. et al. (1991). Adenovirus-mediated transfer of a recombinant alpha 1-antitrypsin gene to the lung epithelium in vivo. Science 252:431-434.
Ruoslahti, E. and Rajotte, D. (2000). An address system in the vasculature of normal tissues and tumors. Ann Rev Immunol 18:813-827.
Rusinko et al. (1989). J Chem Inf Comput Sci 29:251.
Sambrook, J. and Russell, D. W. (2001). Molecular Cloning: A Laboratory Manual, 3rd Ed., Cold Spring Harbor Laboratory, Cold Spring Harbor, N.Y.
Sambrook, J. et al. (1989). Molecular Cloning: A Laboratory Manual, 2nd Ed., Cold Spring Harbor Laboratory, Cold Spring Harbor, N.Y.
Shenk, T. et al. (1984). Genetic analysis of adenoviruses. Curr Top Microbiol Immunol 111:1-39.
Smith, B. F. et al. (1990). Genes transferred by retroviral vectors into normal and mutant myoblasts in primary cultures are expressed in myotubes. Mol Cell Biol 10:3268-3271.
Stevenson, S. C. et al. (1995). Human adenovirus serotypes 3 and 5 bind to two different cellular receptors via the fiber head domain. J Virol 69:2850-2857.
Stevenson, S. C. et al. (1997). Selective targeting of human cells by a chimeric adenovirus vector containing a modified fiber protein. J Virol 71:4782-4790.
Szoka, F., Jr. et al. (1980). Comparative properties and methods of preparation of lipid vesicles (liposomes). Ann Rev Biophys Bioeng 9:467-508.
Takala, A. J. et al. (1996). Time course of beta 2-integrin CD11b/CD18 (Mac-1, alpha M beta 2) upregulation on neutrophils and monocytes after coronary artery bypass grafting. CD11b upregulation after CABG surgery. Scand J Thorac Cardiovasc Surg 30:141-148.
Tripathy, S. K. et al. (1996). Immune responses to transgene-encoded proteins limit the stability of gene expression after injection of replication-defective adenovirus vectors. Nat Med 2:545-550.
Wagner, E. (1992). Coupling of adenovirus to transferrin-polylysine/DNA complexes greatly enhances receptor-mediated gene delivery and expression of transfected genes. Proc Natl Acad Sci USA 89:6099-6103.
Wagner, E. et al. (1990). Transferrin-polycation conjugates as carriers for DNA uptake into cells. Proc Natl Acad Sci USA 87:3410-3414.
Weir, D. M. and Blackwell, C. C. (eds.) (1986). Handbook of Experimental Immunology, Vols. I-IV.
Wickham, T. J. et al. (1997). Targeting endothelium for gene therapy via receptors up-regulated during angiogenesis and inflammation. Cancer Immunol Immunother 45:149-151.
Wolfe, H. R. et al. (2002). In vivo imaging of human colon cancer xenografts in immunodeficient mice using a guanylyl cyclase C-specific ligand. J Nucl Med 43:392-399.
Wolff, M. E. (ed) (1995). Burger's Medicinal Chemistry and Drug Discovery, 5^{th} Ed., Vols. 1-3, John Wiley & Sons, New York.
Wu, G. Y. and Wu, C. H. (1988). Receptor-mediated gene delivery and expression in vivo. J Biol Chem 263:14621-14624.
Wu, C. H. et al. (1989). Targeting genes: delivery and persistent expression of a foreign gene driven by mammalian regulatory elements in vivo. J Biol Chem 264:16985-16987.
Wu, G. Y. et al. (1991). Receptor-mediated gene delivery in vivo. Partial correction of genetic analbuminemia in Nagase rats. J Biol Chem 266:14338-14342.
Wu et al. (eds.). Methods in Enzymology, Vols. 154 and 155.
Xia, D. et al. (1994). Crystal structure of the receptor-binding domain of adenovirus type 5 fiber protein at 1.7 A resolution. Structure 2:1259-1270.
Xiao, X. et al. (1996). Efficient long-term gene transfer into muscle tissue of immunocompetent mice by adeno-associated virus vector. J Virol 70:8098-8108.
Yang, Z. H. et al. (1991). Different interactions of platelets with arterial and venous coronary bypass vessels. Lancet 337:939-943.
Yang, Y. et al. (1996). Immune responses to viral antigens versus transgene product in the elimination of recombinant adenovirus-infected hepatocytes in vivo. Gene Ther 3:137-144.
Zalipsky, S. (1993). Synthesis of an end-group functionalized polyethylene glycol-lipid conjugate for preparation of polymer-grafted liposomes. Bioconjugate Chem 4:296-299.
Zalipsky, S. (1995). In Stealth Liposomes, Lasic, D. and Martin, F. (Eds.), Chapter 9, CRC Press, Boca Raton, Fla.
Zalipsky, S. et al. (1994). Long circulating, cationic liposomes containing amino-PEG-phosphatidylethanolamine. FEBS Lett 353:71-74.
Zalipsky, S. et al. (1995). Peptide attachment to extremities of liposomal surface grafted PEG chains: preparation of the long-irculating form of larninin pentapeptide, YIGSR. Bioconjugate Chemistry 6:705-708.
Zalipsky, S. et al. (1997). Poly(ethylene glycol)-grafted liposomes with oligopeptide or oligosaccharide ligands appended to the termini of the polymer chains. Bioconjugate Chemistry 8:111-118.
Zekorn, T. et al. (1995). Mixed lymphocyte islet culture for assessment of immunoprotection by islet microencapsulation. Transplant Proc 27:3362-3363.
Zenke, M. et al. (1990). Receptor-mediated endocytosis of transferrin-polycation conjugates: an efficient way to introduce DNA into hematopoietic cells. Proc Natl Acad Sci USA 87:3655-3659.Wickham, T. J. et al. (1993). Integrins alpha v beta 3 and alpha v beta 5 promote adenovirus internalization but not virus attachment. Cell 73:309-319.
U.S. Pat. App. No. 60/353,177, filed Feb. 4, 2002
U.S. Pat. App. No. 60/391,967 filed Jun. 26, 2002
U.S. Pat. No. 4,472,509.
U.S. Pat. No. 5,013,556.
U.S. Pat. No. 5,021,236.
U.S. Pat. No. 5,380,830.
U.S. Pat. No. 5,501,979.
U.S. Pat. No. 5,527,528.
U.S. Pat. No. 5,395,619.
U.S. Pat. No. 5,543,328.
U.S. Pat. No. 5,631,018.
U.S. Pat. No. 5,731,190
U.S. Pat. No. 5,756,086.
U.S. Pat. No. 5,844,077.
U.S. Pat. No. 5,922,315
U.S. Pat. No. 5,962,311
U.S. Pat. No. 5,985,655.
U.S. Pat. No. 5,998,205.
U.S. Pat. No. 6,004,798.
U.S. Pat. No. 6,057,155.
U.S. Pat. No. 6,127,525
U.S. Pat. No. 6,153,435
U.S. Pat. No. 6,277,633.
U.S. Pat. No. 6,316,024.
U.S. Pat. No. 6,329,190.
PCT Publication No. WO 94/11524.
PCT Publication No. WO 96/17053.
PCT Publication No. WO 98/07877.
PCT Publication No. WO 98/44938.
PCT Publication No. WO 98/51700.
PCT Publication No. WO 99/25860.
PCT Publication No. WO 00/06195.
PCT Publication No. WO 01/44458.
PCT Publication No. WO 01/92299
PCT Publication No. WO 02/29072.
PCT Publication No. WO 02/067861.

1. A peptide selected from the group consisting of:
(a) CPDLHHHMC (SEQ ID NO:1), CLGQHAFTC (SEQ ID NO:2), CSSNTAPHC (SEQ ID NO:3), CHVLPNGNC (SEQ ID NO:4), CKPQYPSLC (SEQ ID NO:5), CQTARTPAC (SEQ ID NO:6), CNQSQPKHC (SEQ ID NO:7), CTPSKISVC (SEQ ID NO:8), CVSPGPRLC (SEQ ID NO:9), CYALSGVPC (SEQ ID NO:10), CKHPPQPFC (SEQ ID NO:11), CHQSKPLLC (SEQ ID NO:12), CPGPFSNWC (SEQ ID NO:13), CPHKTHLPC (SEQ ID NO:14), CVFPLSHYC (SEQ ID NO:15), CNMIAPSSC (SEQ ID NO:16), CTLGMQFQC (SEQ ID NO:17), CTNPTGMLC (SEQ ID NO:18), CSNMAPRSC (SEQ ID NO:19), CSMAPNMSC (SEQ ID NO:20), CSDLTMEAC (SEQ ID NO:21), CPWPYKYSC (SEQ ID NO:22), CFGGNFHRC (SEQ ID NO:23), CLTTSQQTC (SEQ ID NO:24), CTANSGSFC (SEQ ID NO:25), CQEPLDESC (SEQ ID NO:26), CQMSMFARC (SEQ ID NO:27), CPLTPKAYC (SEQ ID NO:28), CNNSHTALC (SEQ ID NO:29), CLSSDITLC (SEQ ID NO:30), CLTHGPKYC (SEQ ID NO:31), CLGKDLRTC (SEQ ID NO:32), CAPKTHPLC (SEQ ID NO:33), CPTGLMKYC (SEQ ID NO:34), CTWKAPLQC (SEQ ID NO:35), CSHILGPSC (SEQ ID NO:36), CLSTSQYSC (SEQ ID NO:37) or CXXPTPPXC (SEQ ID NO:44);
(b) amino acids 1-8 of a peptide according to (a);
(c) amino acids 2-9 of a peptide according to (a); and
(d) amino acids 2-8 of a peptide according to (a).

2. The peptide of claim 1 selected from the group consisting of CPDLHHHMC (SEQ ID NO:1), CLGQHAFTC (SEQ ID NO:2), CSSNTAPHC (SEQ ID NO:3), CHVLPNGNC
(SEQ ID NO:4), CKPQYPSLC (SEQ ID NO:5), CQTARTPAC (SEQ ID NO:6) and CXXPTPPXC (SEQ ID NO:44)

3. A conjugate of the endothelial cell targeting peptide of claim 1 and a biological agent.

4. The conjugate of claim 3, wherein said biological agent is selected from the group consisting of drugs, peptides, proteins, radionuclides, nucleic acids, gene delivery vectors and liposomes.

5. The conjugate of claim 3, wherein said biological agent is a gene delivery vector selected from the group consisting of SV40 virus, bovine papilloma virus, adenovirus, adeno-associated virus and herpes simplex virus.

6. The conjugate of claim 5, wherein said gene delivery vector is an adenovirus.

7. The conjugate of claim 6, wherein said adenovirus comprises a nucleic acid encoding a fiber protein modified to include said endothelial cell targeting peptide.

8. The conjugate of claim 4, wherein said biological agent is a gene delivery vector which is a retrovirus.

9. The conjugate of claim 8, said retrovirus comprises a nucleic acid encoding a surface protein modified to include said endothelial cell targeting peptide.

10. The conjugate of claim 4, wherein said protein is a growth factor or growth factor fragment.

11. A viral vector comprising a nucleic acid encoding a protein modified to include the peptide of claim 1.

12. The viral vector of claim 11, wherein the vector is derived from a virus selected from the group consisting of SV40 virus, bovine papilloma virus, adenovirus, adeno-associated virus and herpes simplex virus.

13. The viral vector of claim 12, wherein said virus is an adenovirus.

14. The viral vector of claim 13, wherein said modified protein is a fiber protein.

15. The viral vector of claim 12, wherein said virus is a retrovirus.

16. The viral vector of claim 15, wherein the modified protein is a surface protein.

17. A viral vector particle comprising a protein modified to include the peptide of claim 1.

18. The viral vector particle of claim 17, wherein the vector particle is derived from a virus selected from the group consisting of SV40 virus, bovine papilloma virus, adenovirus, adeno-associated virus and herpes simplex virus.

19. The viral vector particle of claim 18, wherein said virus is an adenovirus.

20. The viral vector particle of claim 19, wherein said modified protein is a fiber protein.

21. The viral vector particle of claim 19, wherein said modified protein is sCAR.

22. The viral vector of claim 17, wherein the vector is derived from a retrovirus.

23. The viral vector of claim 22, wherein the modified protein is a surface protein.

24. A nucleic acid encoding a modified viral protein comprising a peptide according to claim 1.

25. The nucleic acid of claim 24, wherein the viral protein is a protein derived from a virus selected from the group consisting of SV40 virus, bovine papilloma virus, adenovirus, adeno-associated virus and herpes simplex virus.

26. The nucleic acid of claim 25, wherein the viral protein is a fiber protein.

27. The nucleic acid of claim 24, wherein the viral protein is a protein derived from a retrovirus.

28. The nucleic acid of claim 27, wherein the viral protein is a surface protein.

29. A nucleic acid encoding a fusion protein comprising a peptide according to claim 1 and a biologically active peptide or protein.

30. The nucleic acid of claim 29, wherein said biologically active peptide or protein is selected from growth factors, toxins, angiogenic peptides, antiangiogenic peptides and pro-apoptotic peptides.

31. A fusion protein comprising a peptide according to claim 1 and a biologically active peptide or protein.

32. The fusion protein of claim 31, wherein said biologically active peptide or protein is selected from growth factors, toxins, angiogenic peptides, antiangiogenic peptides and pro-apoptotic peptides.

33. The conjugate of claim 4, wherein said biological agent is a drug which is a cytotoxic agent.

34. A pharmaceutical composition comprising the conjugate of any oie of claim&-and a pharmaceutically acceptable carrier.

35. A method of targeting a therapeutic substance to endothelial cells which comprises administering the pharmaceutical composition of claim 34.
